SEQUENCE LISTING (11 sequences)

SEQ ID NO: 1 - DNA, 865 bp, Homo sapiens
  ref <308>: DATABASE ACCESSION NUMBER: NM_001549.2
  ref <309>: DATABASE ENTRY DATE: 2008-09-14
  ref <313>: RELEVANT RESIDUES IN SEQ ID NO: (121)..(721)
ctcaaacaccgcctgctaaaaatacccgactggaggagcataaaagcgcagccgagcccagcgccccgcacttttctgagcagacgtccagagcagagtcagccagcatgaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtctcggagatccggcacactgcggaccgctggcgcgtgtccctggatgtcaaccacttcgccccggacgagctgacggtcaagaccaaggatggcgtggtggagatcaccggcaagcacgaggagcggcaggacgagcatggctacatctcccggtgcttcacgcggaaatacacgctgccccccggtgtggaccccacccaagtttcctcctccctgtcccctgagggcacactgaccgtggaggcccccatgcccaagctagccacgcagtccaacgagatcaccatcccagtcaccttcgagtcgcgggcccagcttgggggcccagaagctgcaaaatccgatgagactgccgccaagtaaagccttagcctggatgcccacccctgctgccgccactggctgtgcctcccccgccacctgtgtgttcttttgatacatttatcttctgtttttctcaaataaagttcaaagcaaccacctgtaaaaaaaaaaaaaaaaaa

SEQ ID NO: 2 - DNA, 20 bp, Homo sapiens
agcccgagctgggaaccatt

SEQ ID NO: 3 - DNA, 20 bp, Homo sapiens
aatggttcccagctcgggct

SEQ ID NO: 4 - DNA, 19 bp, Homo sapiens
ccgcagagcgtttgagtat

SEQ ID NO: 5 - DNA, 19 bp, Homo sapiens
atactcaaacgctctgcgg

SEQ ID NO: 6 - DNA, 20 bp, Homo sapiens
gctcaatccgagagagaata

SEQ ID NO: 7 - DNA, 20 bp, Homo sapiens
tattctctctcggattgagc

SEQ ID NO: 8 - DNA, 21 bp, Homo sapiens
aaggacgagcatggctacatc

SEQ ID NO: 9 - DNA, 21 bp, Homo sapiens
gatgtagccatgctcgtcctt

SEQ ID NO: 10 - DNA, 21 bp, Homo sapiens
cacagccgcctcttcgatcaa

SEQ ID NO: 11 - DNA, 21 bp, Homo sapiens
ttgatcgaagaggcggctgtg

(19) United States
(12) Patent Application Publication
(10) Pub. No.: US 2014/0351961 A1
(43) Pub. Date: Nov. 27, 2014

(54) COMPOSITIONS AND METHODS FOR TREATMENT OF METASTATIC CANCER

(71) Applicant: Alexzander A. Asea, Atlanta, GA (US)

(72) Inventor: Alexzander A. Asea, Atlanta, GA (US)

(73) Assignee: Alexzander A. Asea, Atlanta, GA (US)

(21) Appl. No.: 14/241,327

(22) PCT Filed: Aug. 30, 2012
(86) PCT No.: PCT/US12/53237

§ 371 Date: Feb. 26, 2014

Related U.S. Application Data
(60) Provisional application No. 61/529,301, filed on Aug. 31, 2011.

(51) Int. Cl. C12N 15/113; A61K 45/06; A61K 31/713; A61K 31/4375; A61K 47/48; A61K 31/282

(52) U.S. Cl. CPC C12N 15/113; A61K 47/48246; A61K 31/282; A61K 31/713; A61K 31/4375; A61K 45/06; C12N 2310/14; C12N 2310/531; C12N 2320/30
USPC 800/8; 514/44 A; 424/649; 424/93.7; 424/93.71; 536/24.5; 530/402; 530/391.7; 530/394; 424/450; 435/320.1; 435/325

(57) ABSTRACT

Disclosed are methods and compositions for treating cancer that involved an isolated double stranded ribonucleic acid (dsRNA) molecule that inhibits the expression of Hsp-27.

### BACKGROUND OF THE INVENTION

1. Field of the Invention

The present disclosure relates generally to the fields of oncology and molecular biology. More particularly, the invention relates to methods and compositions for treatment of cancer that involve targeting of heat shock protein-27 (Hsp-27).

2. Background

Heat shock proteins (Hsp) are highly conserved proteins found in all prokaryotes and eukaryotes. A wide variety of stressful stimuli, such as for example environmental (U.V. radiation, heat shock, heavy metals and amino acids), pathological (bacterial, parasitic infections or fever, inflammation, malignancy or autoimmunity) or physiological stresses (growth factors, cell differentiation, hormonal stimulation, or tissue development), induce a marked increase in intracellular Hsp synthesis which is known as the stress response. This is achieved by activating the trimerization and nuclear translocation of cytoplasmic heat shock factor-1 (HSF-1) to the heat shock element (HSE) within the nucleus and consequent transcription of Hsp. By binding unfolded, misfolded or mutated peptides or proteins and transporting them to the endoplasmic reticulum (ER), Hsp prevents potential aggregation and/or death. Recently, an additional role has been ascribed to Hsp as danger signals produced and released when cells are under stress and as activators of the immune system. The stress response is designed to enhance the ability of the cell to cope with increasing concentrations of unfolded or denatured proteins.

Based on their apparent molecular mass, Hsp are subdivided into two main groups, the small and large Hsp. Hsp25, the murine homologne of human Hsp27, is a ubiquitously expressed member of the small Hsp family that has been implicated in various biological functions. In contrast to large Hsp, Hsp25/27 act through ATP-independent mechanisms and in vivo they act in concert with other chaperones by creating a reservoir of folding intermediates. Hsp25/Hsp27 are associated with estrogen-responsive malignancies and are expressed at high levels in biopsies as well as circulating in the serum of breast cancer patients. Tumor-host interactions play an important role in determining tumor progression, especially in cases that involve metastasis. Biological response modifiers such as Hsp have been shown to orchestrate some of these events. Thus, it would be desirable to develop a composition and method for the regulation of Hsp expression that can be applied in the treatment and prevention of hyperproliferative diseases such as cancer.

### SUMMARY OF THE INVENTION

The present embodiments are based in part on the finding that double-stranded RNA (dsRNA) molecules that inhibit the expression of heat shock protein 27 (Hsp-2) are highly effective against particular cancer types. For example, the inventor has found that such dsRNA are more effective against highly metastatic breast cancer and pancreatic cancer than non-metastatic or weakly metastatic cancers. In addition, the invention is based in part on the funding that such dsRNA when used in combination with chemotherapy will reduce the toxicity associated with chemotherapy by reducing the required dose of chemotherapy while maintaining superior anti-cancer treatment. For example, the inventor has found that such dsRNA in combination with platinum-containing chemotherapy will reduce the dose of chemotherapy required to eradicate cancer and by extension the chemotherapy-associated side effects. Further, the invention is based on the finding that such dsRNA in combination with topoisomerase 1 inhibitors is highly effective against highly metastatic disease.

In some embodiments, there are compositions comprising a nucleic acid molecule that contains a sequence that is capable of hybridizing under stringent conditions to a human Hsp-27 mRNA, whose cDNA sequence is SEQ ID NO: 1 (NM 001540, which is hereby incorporated by reference). In certain embodiments, the nucleic acid is at least or at most 12, 13, 14,15, 16, 17, 18, 19, 20, 21, 22, 23, 24, 25, 26, 27, 28, 29, 30, 31, 32, 33, 34, 35, 36, 37, 38, 39, 440, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125 nucleotides in length, or any range derivable therein. A nucleic acid molecule may be single-stranded or it may be double-stranded. As a double-stranded molecule, the nucleic acid molecule may include two separate strands or the molecule may be a hairpin in which the two strands are continuous with one another.

Moreover, in some embodiments, the nucleic acid molecule is or comprises RNA. In other embodiments, the nucleic acid molecule is or comprises DNA. In other embodiments, the nucleic acid molecule includes one or awe nucleic acid analogs or modifications.

In some embodiments, a double-stranded molecule is blunt-ended on one end or at least one end. In other embodiments, a double-stranded nucleic acid molecule is blunt-ended on both ends. In specific .embodiments, there may be an overhang on one end or both ends of a double-stranded nucleic acid molecule. The overhang at one end or both ends may be 1, 2, 3, 4, 5, 6, 7, 8, 9, 10 nucleotides or any range derivable therein. If on one end, it may be on the 5′ end of the sense strand or the 3′ end of the sense strand, or It may be on the 5′ end. of the and sense strand or on the 3′ end of the antisense strand.

Embodiments may concern a-nucleic-acid molecule that has at least one strand that i-s 80, 81, 82, 83, 84, 85, 86, 87, 88, 89, 90, 91, 92, 93, 94, 95, 96, 97, 98, 99, or 100% identical to the complement of a contiguous region of SEQ ID NO:1. It is contemplated that such nucleic acids are capable of specifically hybridizing to the contiguous region of SEQ ID NO:1 so as to inhibit expression of Hsp-27 in a human cell. In the ease of double-stranded nucleic acid molecules, it is further contemplated feat there is also a strand that is 80, 81, 82, 83, 84, 85, 86, 87, 88, 89, 90, 91, 92, 93, 94, 95, 96, 97, 98, 99 or 100% identical to a contiguous region of SEQ ID NO:1. The contiguous regions of SEQ ID NO:1 may be a region that constitutes 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 1.05, 106, 107, 108, 109, 110, 111, 112, 113, 114, 115, 116, 117, 118, 119, 120, 121, 122, 123, 124, or 125 contiguous nucleic acid residues of SEQ ID NO:1 (or any range -derivable therein).

In specific embodiments, a nucleic acid molecule, whether single-stranded or double-stranded comprises a strand whose sequence is 80, 81, 82, 83, 84, 85, 86, 87, 88, 89, 90, 91, 92, 93, 94, 95, 96, 97, 98, 99, or 100% identical (or any range derivable therein) to SEQ ID NO:3 (AATGGTTCCCAGCTCGGGCT), SEQ ID NO:5 (ATACTCAAACGCTCTGCGG), SEQ ID NO:7 (TATTCTCTCTCGGATTGAGC); or SEQ ID NO: 9 (GATGTAGCCATGCTCGTCCTT); SEQ ID NO:11 (TFGATCGAAGAGGCGGCTGTG). With double-stranded nucleic acid molecules, one of the strands may have a sequence dial is 80, 81, 82, 83, 84, 85, 86, 87, 88, 89, 90, 91, 92, 93,94, 95, 96, 97, 98, 99, or 100%-identical (or any range derivable therein) to SEQ ID NO:2 (AGCCCGAGCTGGGAACCATT); SEQ ID NO:4(CCGCAGAGCGTTTGAGTAT); SEQ ID NO:6 (GCTCAATCCGAGAGAGAATA); SEQ ID NO:8 (AAGGACGAGCATGGCTACATC); or SEQ ID NO:10 (CACAGCCGCCTCTTCGATCAA). It is specifically contemplated for any SEQ ID NO described above or herein that a corresponding RNA sequence may be used in embodiments instead of the DNA sequence.

It is specifically contemplated that embodiments may involve a double-stranded RNA molecule that comprises the RNA equivalents of SEQ ID NO:2 and SEQ ID NO:3 (referred to as “dsRNA SEQ ID NO:2/SEQ ID NO:3”). Additional embodiments may involve a double-stranded RNA molecule that comprises the RNA equivalents of SEQ ID NO:4 and SEQ ID NO:5 (referred to as “dsRNA SEQ ID NO:4/SEQ ID NO:5”). Further embodiments may involve a double-stranded RNA molecule that comprises the RNA equivalents of SEQ ID NO:6 and SEQ ID NO:7 (referred to as “dsRNA SEQ ID NO:6/SEQ ID NO:7”). Additional embodiments may involve a double-stranded RNA molecule that comprises the RNA equivalents of SEQ ID NO:8 and SEQ ID NO:9 (referred to as “dsRNA SEQ ID NO:8/SEQ ID NO:9”). Certain embodiments may involve a double-stranded RNA molecule that comprises the RNA equivalents of SEQ ID NO:10 and SEQ ID NO:11 (referred to as “dsRNA SEQ ID NO:10/SEQ ID NO:11”).

In some compositions and some methods, there may be more nucleic acid molecules targeting more than one sequence of Hsp-27. In some embodiments, there a combination of different nucleic acid molecules. In some embodiments, there is a combination of nucleic acid molecules that target SEQ ID NO:8 and SEQ ID NO:10. In further embodiments, the combination includes a dsRNA that targets SEQ ID NO:8 and a dsRNA that targets SEQ ID NO:10. In specific embodiments, the combination includes one or more of dsRNA SEQ ID NO:2/SEQ ID NO:3, dsRNA SEQ ID NO:4/SEQ ID NO:5, ds RNA SEQ ID NO:6/SEQ ID NO:7, dsRNA SEQ ID NO:8/SEQ ID NO:9, and/or dsRNA SEQ ID NO:10/SEQ ID NO:11. In particular embodiments, the combination of dsRNA SEQ ID NO:8/SEQ ID NO:9 and dsRNA SEQ ID NO:10/SEQ ID NO:11 are used.

Thus, certain embodiments of the present Invention are directed to methods of treating a subject with, metastatic cancer or at risk of developing metastatic cancer that involve administering to a subject with metastatic cancer or at risk of developing a metastatic cancer a pharmaceutically effective amount of a composition, comprising an isolated double stranded ribonucleic acid (dsRNA) molecule that inhibits the expression of heat shock protein-27 (Hsp-27), The subject can be any subject. For example, the subject may be a mammalian subject such as a mouse, a rat, a rabbit, a dog, a cat, a horse, a cow, a goat, or a primate. In particular aspects the subject is a human subject. The subject may be a subject that has been diagnosed with a tumor. The tumor may be a cancer. For example, the cancer may be brain cancer, ocular cancer, head and neck cancer, skin cancer, lung cancer, esophageal cancer, pancreatic cancer, stomach cancer, liver cancer, prostate cancer, colon cancer, rectal cancer, breast cancer, ovarian cancer, uterine cancer, cervical cancer, lymphoma, leukemia, or testicular cancer.

In particular embodiments, the subject has breast cancer. In more particular embodiments, the breast cancer ER-positive, PgR-positive and Her2-neu-negative. In other embodiments, the breast cancer is ER-negative, PgR-negative, and HER2/neu-positive. The subject may be a subject that has a breast cancer or that has previously been treated for a breast cancer wherein the breast cancer has undergone metastasis.

In other embodiments, the subject has pancreatic cancer or has been previously treated for pancreas cancer. In some embodiments, the subject has metastatic pancreatic cancer.

In some embodiments, the dsRNA has a length of from if 19 to 28 nucleotides. In certain embodiments, one or both strands is 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125 nucleotides in length, or any range derivable therein.

A nucleic acid molecule may have one strand that includes the DNA sequence (or corresponding RNA) as set forth in any of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11. Additional information concerning the dsRNA contemplated for application in the present invention can be found in the specification below and in U.S. Patent Application Pub. No. 20100186102, which is herein specifically incorporated by reference in its entirety.

In some embodiments, the subject is administered a DNA molecule that encodes a strand of a dsRNA molecule as set forth herein.

The dsRNA may optionally be comprised in a vector. Vectors for delivery of nucleic acid molecules are well known to those of ordinary skill in the art For example, the vector may include a cell a liposome, a lipid, or a virus. Nonlimiting examples of viral vectors include adenoviral vectors, retroviral vectors, and lentiviral vectors.

Other aspects concern methods of treating a subject with cancer that involve administering to a subject with cancer a pharmaceutically effective amount of a composition comprising an isolated dsRNA molecule that inhibits the expression of Hsp-27 and a platinum-containing chemotherapeutic agent. Non-limiting examples of platinum-containing chemotherapeutic agents include cisplatin, carboplatin, and oxaliplatin. The dsRNA and the platinum-containing chemotherapeutic agent may be administered concurrently or consecutively. In some embodiments, they are administered in a single pharmaceutically effective composition, and in other embodiments they are administered separately (in separate compositions). The subject may have any type of cancer but in specific embodiments the cancer is breast cancer or pancreatic cancer. In some embodiments, the subject has a primary cancer that has undergone metastasis. For example, the primary tumor may be a breast cancer or a pancreatic cancer. In some embodiments, the subject is administered a nucleic acid encoding one strand of a dsRNA as set forth herein. In specific embodiments, the dsRNA has a length of from 19 to 28 consecutive nucleotides and wherein one strand of the dsRNA comprises SEQ ID NOs: 3, 5, 7, 9, or 11.

Further embodiments concern methods of treating a subject with cancer that involve administering to a subject with cancer a pharmaceutically effective amount of a composition comprising an isolated dsRNA molecule that inhibits the expression of Hsp-27 and a topoisomerase 1 inhibitor. In some embodiments, the subject has a primary cancer that has undergone metastasis or has been previously treated for a primary cancer but now demonstrates evidence of metastatic cancer. In specific embodiments, the cancer is breast cancer or pancreatic cancer. Non-limiting examples of topoisomerase 1 inhibits include irinotecan, topotecan, camptothecin, and lamellarin D. In some embodiments, the subject is administered a nucleic acid encoding one strand of a dsRNA as set forth herein. In specific embodiments, the dsRNA has a length of from 19 to 28 consecutive nucleotides and wherein one strand of the dsRNA comprises SEQ ID Nos: 3, 5, 7, 9, or 11.

Other aspects concern methods of reducing the chemotoxicity of a chemotherapeutic agent that involve administering to a subject with cancer a pharmaceutically effective amount of a composition comprising an isolated dsRNA molecule that inhibits the expression of Hsp-27 concurrently with or prior to administration of a chemotherapeutic agent. In some embodiments, the chemotherapeutic agent is a platinum-containing chemotherapeutic agent selected from the group consisting of cisplatin, carboplatin, and oxaliplatin.

In some embodiments, methods concern giving the chemotherapeutic agent first. In other methods the chemotherapeutic agent is given after the nucleic acid molecule, in certain embodiments, the chemotherapeutic agent is given with 1, 2, 3, 4, 5, 6, 7, 8, 9, 10, 11, 12, 13, 14, 15, 16, 17, 18, 19, 20, 21, 22, 23, 24 hours and/or 1, 2, 3, 4, 5, 6, and/or 7 days before or within the time the nucleic acid molecule is administered to a subject. It is specifically contemplated that in some embodiments exclude methods involving a subject who is given chemotherapy more than 1, 2, 3, 4, 5, 6, 7, 8, 9, 10, 11, 12 months of more prior to being given a nucleic acid molecule. Alternatively, in some embodiments, a patient who previously received chemotherapy but has a recurrent cancer or a cancer deemed unsuccessfully treated by the chemotherapy may be subject to treatment methods involving nucleic acids molecules as described herein.

Embodiments also concern compositions comprising an isolated dsRNA molecule that inhibits the expression of Hsp-27 that has a length of from 19 to 28 consecutive nucleotides and a platinum-containing chemotherapeutic agent, wherein one strand of the dsRNA comprises SEQ ID Nos: 3, 5, 7, 9, or 11. In some embodiments, the chemotherapeutic agent is a platinum-containing chemotherapeutic agent selected from the group consisting of cisplatin, carboplatin, and oxaliplatin.

Further embodiments concern compositions that include 1) an isolated dsRNA molecule that inhibits the expression of Hsp-27 and that has a length of 19 to 28 consecutive nucleotides and 2) a toposisomerase 1 inhibitor. In some embodiments, the composition includes a dsRNA molecule in which one strand of the dsRNA comprises SEQ ID Nos: 3, 5, 7, 9, or 11. Non-limiting examples of topoisomerase 1 inhibitors include any of those previously set forth.

Any of the dsRNA set forth herein may inhibit expression of a protein encoded by a nucleic acid molecule comprising a sequence set forth in SEQ ID NO: 3, 5, 7, 9, or 11; wherein a first strand of the dsRNA is substantially identical to SEQ ID NO: 3, 5, 7, 9, or 11, respectively, and a second strand is substantially complementary to the first.

The dosage range of the dsRNA set forth heroin may range from 0.001 to 1000 mg/kg. In more particular embodiments, the dosage range is 0.01 to 100 mg/kg. In more particular embodiments the dosage range is 0.5 to 50 mg/kg. Administration may be by any method known to those of ordinary skill in the art, such as intravenously, intrathecally, intratumorally, by inhalation, orally, topically, subdurally, intraperitoneally, and so forth.

Some embodiments of the present invention pertain to methods of treating or preventing cancer in a patient, comprising administering to a patient with known or suspected cancer a pharmaceutically effective amount of a composition that includes stem cells capable of differentiating into CD8+ lymphocytes and a pharmaceutically effective amount of a composition comprising an isolated doable stranded ribonucleic acid (dsRNA) molecule that inhibits the expression of HSP-27.

The stem cells may be any stem cells capable of differentiating into a CD8+ lymphocyte. For examples, the stem cells may be multipotent hematopoietic stem cells. The stem cell may be autologous or allogeneic. They may be derived from any source known to those of ordinary skill in the art. For example, they may be derived from bone marrow, peripheral blood, or umbilical cord blood. The composition comprising stem cells may be administered prior to, concurrently with, or following administration of the composition comprising dsRNA. In some embodiments, the stem cells and dsRNA are formulated in a single pharmaceutically effective composition.

Other embodiments of the present invention pertain to methods of treating or preventing cancer in a patient that involve administering to a patient with cancer or at risk of developing cancer a pharmaceutically effective amount of a composition comprising autologous CD8+ T lymphocytes, wherein the lymphocytes have been contacted with isolated double stranded ribonucleic acid (dsRNA) molecules that inhibits the expression of HSP-27.

In particular embodiments, the patient has been diagnosed with cancer, and the patient is administered a pharmaceutically effective amount of a composition comprising an isolated double stranded ribonucleic acid (dsRNA) molecule that inhibits the expression of HSP-27. This is followed by harvesting of autologous CD8+ cells from the patient. Harvesting may be by any method known to those of ordinary skill in the art, such as by lymph node dissection, plasmapheresis, or bone marrow biopsy. The CD8+ cells are then isolated from said harvested tissue using any method known to those of ordinary skill in the art, The CD8+ cells may optionally be frozen and stored for later administration to the patient. The patient may optionally be administered treatment with a conventional chemotherapeutic agent, followed thereafter by administration of the harvested autologous CD8+ cells.

The method of claim 44. The cancer may be of any type. In particular aspects, the cancer is breast cancer, prostate cancer, uterine cancer, ovarian cancer, head and neck cancer, gastric cancer, brain cancer, or bladder cancer. In a specific example, the cancer is breast cancer and the patient has a mutation of BRCA1 or BRCA2. In more particular embodiments, the cancer is metastatic cancer. In a further embodiment, the cancer is a chemoresistant cancer. The patient may be a patient who has undergone a previous treatment with one or more chemotherapeutic agents. The patient may or may not be immunocomprised, with reduced levels of CD8+ lymphocytes.

Further embodiments concern methods of inducing an immune response in a patient with a chemoresistant cancer that involve administering to a patient with cancer or at risk of developing cancer a pharmaceutically effective amount of CD8+ cells or stem cells capable of differentiating into CD8+ cells, wherein said CD8+ cells or stem cells have been contacted with a composition comprising an isolated double stranded ribonucleic acid (dsRNA) molecule that inhibits the expression of HSP-27. The CD8+ cells may be allogeneic cells or autologous cells. Harvesting of cells may be by any method known to those of ordinary skill in the art. Contacting of the cells with the composition comprising dsRNA may be performed in situ in some embodiments. Storage of the cells by freezing may optionally be performed. The cells may then subsequently be administered to the patient. In particular embodiments, the patient, at the time of administration, has previously undergone one or more rounds of chemotherapy resulting in immunosuppression with reduction in levels of CD8+ cells.

Still further embodiments concern methods of preventing the onset of cancer in a patient at risk for development of cancer that involve administering to the patient a pharmaceutical effective amount of CD8+ cells or stem cells capable of differentiating into CD8+ cells, wherein said CD8+ cells or stem cells have been contacted with a composition comprising an isolated double stranded ribonucleic acid (dsRNA) molecule that inhibits the expression of HSF-27. In particular aspects the patient is administered autologous CD8+ cells. More particularly the cells may be hematopoietic stem cells capable of differentiating into CD8+ cells. In a particular embodiment the patient has not been diagnosed with cancer but has a mutation in BRCA1 or BRCA2.

Also included are pharmaceutical compositions for inducing an immune response in a subject with cancer that include a stem cells capable of differentiating into CD8+ T lymphocytes and an isolated double stranded ribonucleic acid (dsRNA) molecule that inhibits the expression of HSP-27. Other pharmaceutical compositions included in the present invention are compositions that include a CD8+ T lymphocytes and an isolated double stranded ribonucleic acid (dsRNA) molecule that inhibits the expression of HSP-27. The isolated dsRNA may be any of the dsRNA previously set forth.

### BRIEF DESCRIPTION OF THE FIGURES

FIG. 1A-D. Permanent gene silencing and expression of Hsp25shRNA in 4T1 breast adenocarcinoma cells using a lentiviral vector. A, HIV-based lentivirus construct pLVTHM was employed to infect 4T1 cells. Construct contains a 5′-long terminal repeats (LTR), gene encoding GFP as reporter and woodchuck hepatitis virus response element (WPRE) as enhancer of gene expression, placed under the tight control of elongation factor alpha (EF-1α) promoter. The Hsp25shRNA stem loop was placed downstream of the H1 promoter, and the self inactivating (SIN) element was placed downstream of the H1-Hsp25shRNA sequence (top panel). Schematic representation of 4T1-Hsp25shRNA and 4T1-controlshRNA hairpin sequences (bottom panel). B, FACSAria generated histograms of lentivirus infected 4T1 cells showing relative number of cells (ordinate) and GFP intensity (abscissa) of gated wild type 4T1 cells (left histogram), 4T1-Hsp25shRNA cells before sorting (middle panel) and after cell sorting (right panel). Data are representative of three independently performed experiments with similar results. C, Sorted 4T1-controlshRNA (top panels) or 4T1-Hsp25shRNA (bottom panels) cells were imaged using a digital inverted fluorescent microscope. Microprograms are phase contrast (left panels) and fluorescence images (right panels) and was obtained under 40× magnification. Data are representative of five independently performed experiments with similar results. D, Western blot analysis of freshly sorted protein lysates from 4T1-controlshRNA (left lane) and 4T1-Hsp25shRNA cells (right lane), immunoblotted with anti-Hsp25 (top panel) or β-actin (bottom panel). Data are representative of three independently performed experiments with similar results.

FIG. 2A-C. Silencing Hsp25 protein expression enhances prohibitin expression. A, Proteins from 4T1-controlshRNA cells (left panel) or 4T1-Hsp25shRNA cells (right panel) were focused over an IPG pH gradient of 4-7, separated on 8-16% polyacrylamide gradient SDS gel and stained with Bio-Safe Coomassie, Square spot (□) represents Ng,Ng-dimethylarginine dimethylaminohydrolase 2 and prohibitin; circle spot (◯) represents proteasome (prosome, macropain) 28 subunit alpha, PA28α and triangle spot (Δ) represents undetectable proteins, as judged by mass spectrometry. Data is a representative experiment from three independently performed experiments with similar results. B, 4T1-controlshRNA cells (filled bars) and 4T1-Hsp25shRNA cells (open bars) were used to isolate total RNA and the relative prohihitin mRNA expression was measured using real-time PGR analysis. Data are the mean prohibitin mRNA expression ±SD and is the sum of three independently performed experiments. *, p<0.001 vs 4T1-controlshRNA cells (Student's t-test). C, 4T1-controlshRNA cells (left lane) and 4T1-Hsp25spRNA cells (right lane) were lysed, proteins extracted and subjected to immunoblotting with anti-prohlbidn Mab or β-actin (top panel). The intensity of the bands were analyzed by densitometry with a video densitometer (Chemilmager™ 5500; Alpha Innotech, San Leandro, Calif.) using the AAB software (American Applied Biology) (bottom panel). Bars represent the mean prohihitin protein expression and is a representative experiment from three independently performed experiments with similar results.

FIG. 3A-C. Proteasome activity is increased by silencing Hsp25 protein expression. A, 4T1-controlshRNA cells (filled bars) and 4T1-Hsp25shRNA cells (open bars) were used to isolate total RNA and the relative FA28α mRNA expression was measured using real-time PCR analysis. Data are the mean prohibitin mRNA expression ±SD and is the sum of four independently performed experiments. *, p<0.001 vs 4T1-controlshRNA cells (Student's t-test). B, 4T1-controlshRNA cells (left lane) and 4T1-Hsp25shRNA cells (right lane) were lysed, proteins extracted and subjected to immunoblotting with anti-PA28α Mab or β-actin (top panel). The intensity of the bands were analyzed by densitometry with a video densitometer (Chemilmager™ 5500; Alpha Innotech) using the AAB software (bottom panel). Bars represent the mean PA28α protein expression and is a representative experiment from three independently performed experiments with similar results. C, 20S proteasome activity was measured by incubation of cell extracts from 30 μg 4T1-controlshRNA (filled bars) or 4T1-Msp25shRNA (open bars) for 90 min with a fhiorogenie substrate (Sue-LLVY-AMC) in the absence or presence of iactaeystin (25 μM). Free AMC fluorescence was measured by using a 380/460 nm filter set in a fluorometer. Data are the mean proteasonie activity (% control±SD) and is the sum of three independently performed experiments. *, p<0.001 vs 4T1-controlshRNA cells (Student's t-test).

FIG. 4A-D, Silencing hsp25 gene expression in 4T1 cells induces tumor regression. A, 4T1-controlshRNA cells or 4T1-Hsp25shRNA cells were injected into the mammary pads of female BALB/c mice and tumor growth was monitored on specific days post tumor cell injection using the Maestro™ in vivo imaging system (CRI). Data are fluorescence microprogram of GFP-tagged tumors (green fluorescence) measured on various days post tumor cell injection (top panel). Bars represent the mean GFP signal/exposure (total signal sealed counts/seconds) from 4T1-controlshRNA cells (filled bars) or 4T1-Hsp25shRNA cells (open bars) and is the sum of three mice/group (n=3). *, p<0.001 vs 4T1-controlshRNA cells (Student's t-test) (bottom panel). B, 10⁴ 4T1-controlshRNA-e-GFP(+) cells (filled squares) or 4T1-controlshRHA-e-GFP(−) cells (open squares) or 4T1-Hsp25shRNA-e-GFP(+) cells (filled circles) or 4T1-Bsp2SRNA-e-GFP(−) cells (open circles) or 4T1 wt cells (open diamonds) were injected into the mammary pads of female BALB/c wild type mice (left panel) or female BALB/c nude mice (right panel) and tumor growth was monitored on specific days post tenor cell injection using an electronic caliper. Data are mean tumor volume±SD and is a representative experiment from two independently performed experiments (n=5). C, H&E staining of lungs from mice 34 days after TCI; arrow indicates lung micrometastasis. Data is a representative of four independently performed experiments with similar results. D, Colony formation of tumor derived from lungs of mice injected with 4T1-controlshRNA (top panel) or 4T1-Hsp25shRNA cells (bottom panel), was platted at different dilution ratios (1:20-1:320). Plates were stained and the number of cells was counted (top panel). Data represent the mean number of colonies±SD and is a representative experiment from lour independently performed experiments. *, p<0.001 vs 4T1-controlshRNA cells (Student's t-test).

FIG. 5A-F. Silencing hsp25 gene expression augments CB8⁺ T lymphocyte-dependent tumor recognition and killing. A, Female BALB/c mice (6-8 weeks old) were injected i.p., with PBS (black lines) or anti-CD4 (L3T4; blue lines), anti-CD8 (Ly-2; red lines) and anti-NK (5E6; green lines) 4 days before injection of 10⁴ 4T1-controlshRNA cells (left panels) or 10⁴ 4T1-Hsp25shRNA cells (right panels) into the abdominal mammary pads of mice every week. Data represent mean tumor volume (mm³) and is representative of four independently performed experiments (n=3). B, Splenocytes from female BALB/c mice was recovered and CD8⁺T cells isolated using negative selection technique according to the manufacturer's instructions (Milteoyi Biotech), Ceils (10⁶) were stained with 0.5 μg of anti-CD8a (Ly-2), washed and incubated with 0.6 μg of the F(ab)2 anti-rat IgG-FITC (Caltag, Burlingame, Calif., USA) and analyzed by flow cytometry. Samples were acquired in a FACScalibur cytometer and analyzed using the Cell Quest software (Beckton Dickinson, San Jose, Calif., USA). A total of 20,000 cells per condition were recorded and viable cells were defined according to the FSC and SSC pattern. Data are histograms for the relative number of cells expressing CD8a (Ly-2) and is a representative experiments from three independently performed experiments with similar results. C, 4T1-Hsp25shRNA cells (10⁴) were injected into mammary pads of 6-8 week-old female BALB/c mice. When tumors started regressing at the end of two weeks, and spleen tissues were harvested from, the animals and CD8⁺ T cells (filled squares) or CD8⁻ T cells (open squares) were isolated using negative selection technique according to the manufacturer's instructions (Miltenyi Biotech), and admixed with 4T1-controlshRNA-e-GFP(+) cells or 4T1-controlshRNA-e-GFP(−) cells or BNL cells seeded at various effector/target ratios (10:1, 20:1 and 40:1), in quintuplicate in 96-well tissue culture plates. Cytotoxicity was measured by lactate dehydrogenase-cytotoxicity assay kit II, according to the manufacturer's instructions (BioVision). Data are the sum of four independently performed experiments. *, p<0.001 vs CD8⁻ cells (Student's t-test). A 4T1-Hsp25shRNA cells (10⁴) were injected into the mammary glands of female BALB/c mice and tumor regression was measured using Maestero™ in vivo imaging system. At the end of four weeks splenocytes were collected and CD8⁺ T cells were isolated and enriched by negative selection according to manufacturer's instruction (Milteny Biotec). The cells recovered were designated CD8⁺ T cells. The fraction depleted of CD8⁺ T cells were designated CD8⁺ T cells. Adoptive transfer of 10⁶ 4T1-Hsp25shRNA reactive CD8⁺ T cells or CD8⁺ T cells (top panel) was performed via the tail vein on day 5 post TCI Into mice injected with 4T1-controlshRNA tumors. Data are fluorescence micropictogram of GFP-tagged tumors (green fluorescence) measured on various days post tumor cell injection (top panel). Bars represent the mean GFP signal/exposure (total signal scaled counts/seconds) from animals adoptively transferred with CD8⁻ T cells (filled bars) or CD8⁺ T cells (open bars) and is the sum of three mice/group (n=3), *, p<0.001 vs 4T1-controlshRNA cells (Student's t-test) (bottom panel). E, BMDC were recovered from female C57BL/6 (H2^{b}) mice (left panel) or female BALB/c (H2^{d}) mice (right panel) and transteeted with either control-siRNA (open bars) or Hsp25-siRNA (filled squares) and treated with 100 ng/ml OVA peptide (SSL) or 100 ng/ml control peptide (FBI) or 10 μM MG-132. Cells were fixed with paraformaldehyde and admixed with B3Z cells. Bars represent the concentration of IFN-γ released into the supernatant±SD and is the sum of four independently performed experiments. *, p<0.001 vs control-siRNA (Student's t-test). F, On day 0, female BALB/c mice were injected with either 10⁴ 4T1-controlshRNA cells alone (open diamonds) or 4T1-Hsp25shRNA cells alone (open circles) or BNL (open squares). Two additional groups of mice were injected with 4T1-Hsp25shRNA cells. After 60 days, these mice were re-challenged with either 10⁴ 4T1-wt cells (4T1-Hsp25shRNA+4T1-wt; filled circles) or 10⁵ BNL cells (4T1-Hsp25shRNA+BNL; filled squares), and tumor growth was monitored on specific days post tumor cell injection using an electronic caliper. Data are mean tumor volume±SD and is the sum of two independently performed experiment (n+5).

FIG. 6A-C. Effects of gene targeted Hsp25 silencing on 4T1 breast adenocarcinoma cell functions. A, 4T1-controlshRNA cells (filled circles) or 4T1-wt cells (filled diamonds) or 4T1-Hsp25shRNA cells (open circles) were seeded at 104 cells into T-250 tissue culture flasks on day 0 in media containing DMEM supplemented with 10% FBS. At various times cell viability was determined using a hemocytometer under a phase-contrast light microscope (top panel). Data represent the mean number of cells±S.D. and is the sum of four independently performed experiments performed in quadruplicates. Supernatant was also recovered and the percentage of cell death was measured using the CytoTox 96 Non-Radioactive Cytotoxicity Assay according to the manufactures instructions (Promega), the percentage of LDH released versus total LDH was calculated, (bottom panel). Data are mean percentage cell death±SD (n=4) and represent four independently performed experiments. B, 4T1-controlshRNA cells (top panel) or 4T1-Hsp25shRNA cells (bottom panel) were seeded into 6-well tissue culture plates and grew in DMEM complete medium. After cells were grown to conflueney, wounds were made by sterile 10 μl pipette tips. Cells were washed with PBS to remove floating cells and fresh medium with or without 10% FBS was added and incubated at 37° C. in humidified atmosphere with 5% CO₂. After 22 h incubation cells were fixed and photographed under a phase-contrast light microscope. Data are phase-contrast Images (10× field) of the wound healing process and is a representative experiment from three independently performed experiments with similar results, C, 4T1-controlshRNA or 4T1-Hsp25shRNA cells were trypsinized, counted and added to the upper section of the Hoyden chamber according to manufacturer's instruction (BD Biosciences, USA). PBS (1%) was added to die top chamber and 10% PBS added to the lower chamber. Transwell plates were incubated for an additional 20 h at 37° C. Cells on the inside of the transwell inserts were removed with a cotton swab, and cells on the underside of the insert were fixed and stained by using Hema 3 manual staining system (Fisher Scientific). Photographs of ten random fields were taken, and the cells were counted to calculate the mean number of cells that had transinvaded. Data are phase contrast pictograms of 4T1-controlshRNA cells (left panel) or 4T1-Hsp25shRNA cells (right panel) at 40× magnification (upper panels). Bars represent the mean number of invading cells±S.D. and is the sum of triplicate wells, *, p<0.01 vs 4T1-controlshRNA (Student's t-test) (bottom panel).

FIG. 7. Combining CH101 with oxaliplatin synergistically functions to reduce the IC₅₀ in the weakly metastatic pancreatic cell Panc-1. Panc-1 cells (10⁶) were plated in 96-well plates and either pre-treated with control (top panel) or CH101 (bottom panel) for 48 h in a 37 degree C incubator. Panc-1 cells were then treated with various doses of oxaliplatm and further incubated for 72 hours. Cytotoxicity was measured using the classical MTS assay.

FIG. 8A-B. Combining CH101 with oxaliplatm synergistieally functions to reduce the IC₅₀ in the highly agreesive, highly metastatic pancreatic cell, AsPC1, AsPC1 cells (10⁶) were plated in 96-well plates and either pre-treated with control (top panel) or CH101 (bottom panel) for 48 hrs in a 37 degree C incubator. AsPC1 cells were then treated with various doses of oxaliplatm (A) or irlootecan (B) and further incubated for 72 h. Cytotoxicity was measured using the classical MTS assay.

### DETAILED DESCRIPTION

### A. Definitions

The following are to serve as definitions of terms that may be used throughout this disclosure.

A “vector” is a repHcon, such as plasmid, phage, viral construct or cosmid, to which another DNA segment may be attached. Vectors are used to transduce and express the DNA segment in cells. As used herein, the terms “vector”, “construct”, “RNAi expression vector” or “RNAi expression construct” may include replicons such as plasmids, phage, viral constructs, eosniids. Bacterial Artificial Chromosomes (BACs), Yeast Artificial Chromosomes (YACs) Human Artificial Chromosomes (HACs) and the like into which one or more RNAi expression cassettes may be or are ligated.

A “promoter” or “promoter sequence” is a DMA regulatory region capable of binding RNA polymerase in a cell and initiating transcription of a polynucleotide or polypeptide coding sequence such as messenger RNA, ribosomal RNAs, small nuclear or nucleolar RNAs or any kind of RNA transcribed by any class of any RNA polymerase.

The phrase “stringent hybridization conditions” or “stringent conditions” refers to conditions under which au oligomeric compound of the invention will specifically hybridize to its nucleic acid target. Stringent conditions are sequence-dependent and will vary with different circumstances and in the present context; “stringent conditions” under which oligomeric compounds hybridize to a nucleic acid target are determined by the nature and composition of the oligomeric compounds and the assays in which they are being investigated. One having ordinary skill in the art will understand variability in the experimental protocols and be able to determine when conditions are optimal for stringent hybridization with minimal non-specific hybridization events.

“Complementarity,” as used herein, refers to the capacity for precise pairing of one nucleobase with another. For example, if a monomelic subunit at a certain position of an oligomeric compound is capable of hydrogen bonding with a monomelic subunit at a certain position of a nucleic acid target, then the position is considered to be a complementary position. Conversely, a position is considered “non-complementary” when monomelic suhunits are not capable of hydrogen bonding. The oligomeric compound and the target nucleic acid are “substantially complementary” to each other when a sufficient number of complementary positions in each molecule are occupied by rnonomerie suhunits that can hydrogen bond with each other. Thus, the term “substantially complementary” is used to indicate a sufficient degree of precise pairing over a sufficient number of rnonomerie suhunits such that stable and specific binding occurs between the oligomeric compound and a target nucleic acid. The terms “substantially complementary” and “sufficiently complementary” arc herein used interehangably. An oligomeriC compound need not be 100% complementary to that of its target nucleic acid to be specifically hybridizable. Moreover, an oligomeric compound may hybridize over one or more segments such that intervening or adjacent segments are not involved in the hybridization (e.g., a bulge, a loop structure or a hairpin, structure). A “non-complementary nucleobase” means a nucleobase of an aniisense oligonucleotide that is unable to undergo precise base pairing with a nucleobase at a corresponding position in a target nucleic acid. In some embodiments there are non-complementary positions, also known as “mismatches”, between the oligomeric compound and the target nucleic acid, and such non-complementary positions may be tolerated between an oligomeric compound and the target nucleic acid provided that the oligomeric compound remains substantially complementary to the target nucleic acid.

An oligomeric compound and a nucleic acid target are “fully complementary” to each, other when each nucleobase of an oligomeric compound is capable of undergoing basepairing with corresponding positions in a nucleic acid target. As used herein, the term “full length complementarity” means that an oligomeric compound comprises a contiguous sequence of nucleosides with the same length as the target mRNA and is fully complementary to a region of the target mRMA (for example if one region is 22 nucleotides in length, an oligomeric compound with full length complementary oligomeric compound is also 22 nucleotides in length). In some embodiments, an oligomeric compound has full length complementarity to a target mRNA.

A “target region” is defined as a portion of the target nucleic acid having at least one identifiable sequence, structure, function, or characteristic. “Target segments” are defined as smaller or sub-portions of target regions within a target nucleic acid such as the mRNA corresponding to SEQ ID NO:1. The locations on the target nucleic acid to which compounds and compositions of the invention hybridize are herein referred to as “suitable target segments.” As used herein the term “suitable target segment.” is defined as at least a 6-nucleobase portion of a target region to which an oligomeric compound is targeted. In one embodiment, a suitable target segment, of the target mRNA is the seed sequence of the mRNA.

A cell has been “transformed”, “transduced” or “transfected” by an exogenous or heterologous nucleic acid or vector when such nucleic acid has been introduced inside the cell, for example, as a complex with transaction reagents or packaged in viral particles. The transforming DNA may or may not be integrated (covalently linked) into the genome of the cell. With respect to eukaryotic cells, a stably transformed cell is one in which the transforming DNA has become integrated into a host cell chromosome or is maintained extra-chromosonmlly so that the transforming DNA is inherited by daughter cells during cell replication or the transforming DNA is in a non-replicating, differentiated cell in which a persistent episoroe is present,

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The terms “cancer” and “cancerous” refer to or describe the physiological condition, in mammals that is typically characterized by unregulated cell, growth/proliferation. Examples of cancer include, but are not limited to, carcinoma, lymphoma, blastoma, sarcoma, and leukemia, More particular examples of such cancers include squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, brain cancer, endometrial cancer, testis cancer, cholangioearcinoma, gallbladder carcinoma, gastric cancer, melanoma, and various types of head and neck cancer, Dysregulation of anglogenesis can lead to many disorders that can be treated by compositions and methods of the invention. These disorders include both non-nsoplastic and neoplastic conditions. Neoplastic conditions include but are not limited those described above.

“Non-neoplastlc disorders” include but are not limited to undesired or aberrant hypertrophy, arthritis, rheumatoid arthritis (RA), psoriasis, psoriatic plaques, sarcoidosis, atherosclerosis, atherosclerotic plaques, diabetic and other proliferative retinopathies including retinopathy of prematurity, retrolental fibroplasia, neovaseular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovaseular disease, vascular restenosis, arteriovenous malformations (AVM), meningioma, hemangioma, angiofibroma, thyroid hyperplasias (including Grave's disease), corneal, and other tissue transplantation, chronic inflammation, lung inflammation, acute lung injury/ARDS, sepsis, primary pulmonary hypertension, malignant pulmonary effusions, cerebral edema (e.g., associated with acute stroke/closed head injury/trauma), synovial inflammation, pannus formation in RA, myositis ossificans, hypertropic bone formation, osteoarthritis (OA), refractory ascites, polycystic ovarian disease, endometriosis, 3rd spacing of fluid diseases (pancreatitis, compartment syndrome, burns, bowel disease), uterine fibroids, premature labor, chronic inflammation such as IBD (Crohn's disease and ulcerative colitis), renal allograft rejection, Inflammatory bowel disease, nephrotic syndrome, nodesired or aberrant tissue mass growth (non-cancer), hemophilic joints, hypertrophic scars, inhibition of hair growth, Osler-Weber syndrome, pyogenic granuloma retrolental fibroplasias, scleroderma, trachoma, vascular adhesions, synovitis, dermatitis, preeclampsia, ascites, pericardial effusion (such as that associated with pericarditis), and pleural effusion.

“treatment” as used herein refers to clinical intervention in an attempt to alter the natural course of the individual or cell being treated, and can be performed either for prophylaxis or during the course of clinical pathology. Desirable effects of treatment include preventing Occurrence or recurrence of disease, alleviation of symptoms, reduction of any direct or indirect pathological consequences of the disease, decreasing the rate of disease progression, amelioration or palliation of the disease state, and remission or improved prognosis. In some embodiments, antibodies of the invention are used to delay development of a disease or disorder. In non-limiting examples, antibodies of the invention may be used to reduce the rate of tumor growth or reduce the risk of metastasis of a cancer.

An “Individual,” “subject,” or “patient” is a vertebrate, e.g. a mammal, including especially a human. Mammals include, but are not limited to, humans, domestic and farm animals, and zoos, sports, or pet animals, such as dogs, horses, cats, cows, rats, mice, etc.

An “effective amount” refers to an amount effective, at dosages and for periods of time necessary, to achieve the desired therapeutic or prophylactic result.

A “therapeutically effective amount” of a substance/molecule of the invention refers to an amount of a drug effective to treat a disease or disorder in a mammal. It may vary according to factors such as the disease state, age, sex, and weight of the individual, and the ability of the substance/molecule to elicit a desired response in the individual. A therapeutically effective amount is also one in which any toxic or detrimental effects of the substance/molecule are outweighed by the therapeutically beneficial effects.

A “prophylactically effective amount” refers to an amount effective, at dosages and for periods of time necessary, to achieve the desired prophylactic result. As a prophylactic dose is used in subjects prior to or at an earlier stage of disease. The prophylactically effective amount typically, but not necessarily, will be less than the therapeutically effective amount.

A “chemotherapeutic agent” is a chemical compound useful in the treatment of cancer. Non-limiting examples of chemotherapeutic agents include alkylating agents such as thiotepa and CYTOXAN, cyclosphosphamide; alkyl sulfonates such as busulfan, improsulfan and piposulfan; aziridines such as benzodopa, earboquone, meturedopa, and aredopa; ethylenimmes and methylameiamines including altretamine, txiethylenemelarnine, trietyienephosphoramide, triethiylenethiophosphoramide and trimethylolomelamine; TLK 286 (TBLCYTA); aeetogenins (especially bullatacin and bullatacinone); delta-9-tetrahydrocannabinol (dronabinol, MARINOL); beta-lapachone; lapachol; colchicines; betulinic acid; a camptothecin (including the synthetic analogue topotecan (HYCAMTIN), CPT-11 (irinotecan, CAMPTOSAR), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptopbycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofostaraide, uracil mustard; nitrosureas such as carmustine, chlorozotocin, fotemustine, lomustine, nimustine, and ranimnustine; bisphosphonates, such as clodronate; antibiotics such as the enediyne antibiotics (e.g., calicheamicin, especially calicheamicin gamma 11 and calicheamicin omega 1 (see, e.g., Anger, Chem Intl. Ed. Engl., 33: 183-186 (1994)) and anthracyclines such as annamycin, AD 32, alcarubicin, daunornblcin, dexrazoxane, DX-52-1, epiruhicin, GPX-100, idarubicin, KRN5500, menogaril, dynemicin, including dynemicin A, an esperarnidn, neocarzinostatin chromophore and related ehrornoprotein enediyne antiobiotic chromophores, aclacinomysins, actinomycin, authramycin, azaserine, bleomycins, cactinomyein, carabicin, carminomycin, carzinophilin, chromomycinis, dactinomycin, detorubicin, 6-diazo-5-oxo-L-norleucine, ADRIAMYCIN or doxorubicin (including morpholino-doxorubicin, cyanomorpholino-doxorubicin, 2-pyrrolino-doxorubicin, liposomal doxorubicin, and deoxydoxorubicin), esorubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; folic acid analogues ssuch as denopterin, pteropterin, and trimetrexae; purine analogs such as fludarabine, 6-mercaptopurine, thiamiprine, and thiguanine;pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane and trilostate; folic acid replenisher such as folinic acid (leucovorin); aceglatone; anti-folate anti-meoplastic agents such as ALIMTA, LY231514 pemetrexed, dihydrofolate reductase inhibitors such as methotrexate, anti-metabolites such as 5-fluorouracil (5-FU) and its prodrugs such as UFT, S-1 and capecitabine, and thymidylate synthase inhibitors and glycinamnide ribonucleotide formyltransferase inhibitors such as raltitrexed (Tomudex, TDX); inhibitors of dihydrophyrimidine dehydrogenase such as eniluracil; aldophosphamide glycoside; arninolevulinic acid; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitracrine; pentastain; phenamet; pirarubicin; losoxantrone; 2-ethylhydrozide; procarbazine; PSK polysacchande complex (JHS Natural Products, Eugene, Oreg.); razoxane; rhizoxin; sizofiran; spirogermanium; tenuazonic acid; triaziquone; 2,2′,20″-trichlorotriethylamine; trichothcenes (especially T-2 toxin, verracurin A, roridin A and anguidine); urethan; vindesine (ELDISINE, FILDESIN); dacarbazine; mannomustine; mitobronitol; mitolactol; pipobraman; gacytosine; arabinoside (“Ara-C”); cyclophosphamide; thiotepa; taxoids and taxanes, e.g., TAXOL, paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, Ill.) and TAXOTERE or doxetaxel (Rhone-Poulene Rorer, Anthony, France); Chloranbucil; gemcitabine (GEMZAR); 6-thioguanine; mercaptopurine; platinum; platinum anaogs or platinum-based analogs such as cisplatin, oxaliplatin and carboplatin; vinblastine (VELBAN); etoposide (VP-16); ifosfamide; mitoxantrone; vincristine (ONCOVIN); vinca alkaloid; vinorelbme (NAVELBINE); novantrone; edatrexate; daunomycin aminopterin; xeloda; ibandronate; topoisomerase inhibitor RFS 2000; difluoromethylomithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN) combined with 5-FU, leucovorin, and ADCETRIS (Brentuximab Vedotin). It is specifically contemplated that any of the chemotherapeutic agents recited above may be specifically excluded in compositions and methods discussed herein.

Also included in the definition of chemotherapeutic agents are anti-hormonal agents that act to regulate or inhibit hormone action on tumors such as anti-estrogens and selective estrogen receptor modulators (SERMs), including, for example, tamoxifen (including NOLVADEX or tamoxifen), raloxifene, droloxifene, 4-hydroxytamoxifen, trioxifene, keoxifene, LYI17018, onapristone, and FARESTO or toremifene; aromatase inhibitors that inhibit the enzyme aromaiase, which regulates estrogen production in the adrenal glands, such, as, for example, 4(5)-imidazoles, aminoglutethimide, MEGASB or megestrol acetate, AROMASIN or exemesiane, formesianic, fadrozole, RIVISOR or vorozole, FEMARA or letrozole, and ARIMIDEX or anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those that inhibit expression of genes in signaling pathways implicated in adherent cell proliferation, such as, for example, PKC-alpha, Raf, H-Ras, and epidermal growth factor receptor (EGF-R); vaccines such as gene therapy vaccines, for example, ALLOVECTIN or vaccine, LEUVECTIN or vaccine, and VAXID or vaccine; PROLEUKIN or rIL-2; LURTOTECAN or topoisomerase 1 inhibitor; ABARELIX or rmRH; and pharmaccuticaliy acceptable salts, acids or derivatives of any of the above. Also included in this definition are small, molecule toxins, such as a calicheamicin, maytansinoids, dolastatins, aurostatins, a trichothecene, and CC1065.

A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes.

An “isolated” nucleic acid molecule is a nucleic acid molecule chat is identified and separated from at least one contaminant nucleic acid molecule with which it is ordinarily associated in the natural source of the antibody nucleic acid. An isolated nucleic acid molecule is other than In the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule, is in a chromosomal location different from that of natural cells.

“Polynucleotide” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure: may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioatcs, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynueleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylaied or substituted with amines or organic- capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha-anomeric sugars, epimeric sugars such as arabinose, xyloses or lyxoses, pyranose sugars, fluranose sugars, sedoheptuloses, acyclic analogs and a basic nucleoside analogs such as methyl riboside. One or more phospbodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a phage vector. Another type of vector is a viral vector, wherein additional DNA segments may be ligated into the viral genome (such as an adenoviral vector, a lentiviral vector, etc.). Certain vectors are capable of autonomous replication in a host cell into which they are introduced (e.g., bacterial vectors having a bacterial origin of replication and eplsomal mammalian vectors). Other vectors (e.g., non-episomal mammalian vectors) can be integrated into the genome of a host cell upon introduction into the host cell and thereby are replicated along with, the host genome. Moreover, certain vectors are capable of directing die expression of genes to which they are operatively linked. Such vectors are referred to herein as “recombinant expression vectors” (or simply, “recombinant vectors”).

The term “sequence identity” (or “sequence similarity”) is herein defined as a relationship between two or more nucleic acid (polynucleotide) or amino acid (polypeptide) sequences, as determined by comparing the sequences. Usually, sequence identities or similarities are compared, typically over the whole length of the sequences compared. However, sequences may be compared over shorter comparison windows. In the art, “identity” also means the degree of relatedness between nucleic acid or amino acid sequences, as the case may be, as determined by the match between strings of such sequences.

### B. Nucleic Acid Compositions and Methods

Disclosed herein are compositions and methods for selectively reducing the expression of a gene product from a desired targeted gene in a cell or tissue. In an embodiment, the cell is an eukaryotic cell. Also disclosed herein are methods of treating diseases whose coarse or progression are influenced by the expression of the desired targeted gene. More specifically, disclosed herein are compositions and methods for regulating the expression of heat shock proteins (Hsp). Further disclosed herein are methods for the delivery of compositions that regulate the expression of heat shock proteins to cells and tissues.

In some embodiments, these compositions comprise pharmaceutical formulations comprising therapeutic amounts of materials which may be used in the treatment of an organism experiencing a dysfunction, undesirable medical condition, disorder, or disease state. The dysfunction, undesirable medical condition, disorder, or disease state will be collectively referred to hereinafter as an “undesirable condition.” Herein the undesirable condition is one in which the level of expression of an eukaryotie Hsp may contribute to the onset or progression of the undesirable condition and as such the undesirable condition is one which may he amenable to siRNA therapy. Thus, the undesirable condition includes conditions such as “genetic diseases” which refer to conditions attributable to one or more gene defects, “acquired pathologies” which refer to pathological conditions that are not attributable to inborn defects, cancers, diseases, and the like. Herein “treatment” refers to an intervention performed with the intention of preventing the development or altering the pathology of the undesirable condition. Accordingly “treating” refers both to therapeutic treatments and to prophylactic measures. In an embodiment, administration of therapeutic amounts of compositions of the type described herein to an organism confers a beneficial effect on the recipient in terms of amelioration of the undesirable condition. Herein “therapeutic amounts” refers to the amount of the composition necessary to elicit a beneficial effect. Alternatively, the compositions described herein may be used prophylactically for reducing the potential onset or reoccurrence of an undesirable condition in a recipient not currently experiencing an undesirable condition in which the level of Hsp expression contributes to the onset or reoccurrence of said undesirable condition.

In an embodiment, the compositions comprise one or more isolated or purified nucleic acid molecules and methods of utilizing these nucleic acid molecules to reduce the expression of one or more Hsp in a cell. As used herein, the term “nucleic acid molecule” can include DNA molecules; RNA molecules; analogs of a DNA or RNA molecule generated using nucleotide analogs; derivatives thereof or combinations thereof. A nucleic acid molecule may be single-stranded or double-stranded, and the strandedness will depend upon its intended use. Fragments or portions of the disclosed nucleic acid molecules are also encompassed by the present disclosure. By “fragment” or “portion” is meant less than full length of the nucleotide sequence. As used, herein, an “isolated” or “purified” nucleic acid molecule is a nucleic acid molecule that is separated from other nucleic acid molecules that are usually associated with the isolated nucleic acid molecule. Thus, an isolated nucleic acid molecule includes, without limitation, a nucleic acid molecule that is free of sequences that naturally flank one or both ends of the nucleic acid in the genome of the organism from, which the isolated nucleic acid, is derived (e.g., a c-DNA or genomic DNA. fragment produced by PCR or restriction endonnclease digestion). Alternatively, the “isolated” or “purified” nucleic acid molecule may be substantially free of other cellular material or culture medium when produced by recombinant techniques or substantially free of chemical precursors or other chemicals when chemically synthesized. Herein substantially free refers to the level of other components being present in amounts that do not adversely affect the properties of the Hsp reducing compositions and/or the organisms to which the compositions are introduced. For example, the nucleic acid molecules may be greater than about 70% pure, alternatively greater than about 75%, 80%, 85%, 90%, or 95% pure. Such an isolated nucleic acid molecule is generally introduced into a vector (e.g., a cloning vector, or an expression vector, or an expression construct) for convenience of manipulation or to generate a fusion nucleic acid molecule as will be described in more detail later herein. In addition, an isolated nucleic acid molecule can include an engineered nucleic acid molecule such as a recombinant or a synthetic nucleic acid molecule.

A nucleic acid molecule may be used to regulate the expression of one or more cellular proteins. For example, the nucleic acid molecule of this disclosure may function to reduce the expression of one or more Hsp. In an embodiment, the nucleic acid molecules comprise RNA and introduction of the RNA into a cell results in post transcriptional silencing of at least one RNA transcript. The present disclosure provides for such RNA molecules, the DNA molecules encoding such RNA molecules, the polypeptide encoded by such nucleic acid molecules, antibodies raised to said polypeptides; or combinations thereof. The RNA molecules of this disclosure can be used in a variety of forms; nonlimiting examples of which include antisense RNAi and shRNA.

The disclosed methodologies utilize the RNA interference (RNAi) mechanism to reduce the expression of one or more RNA transcripts. The term “RNA interference or silencing” is broadly defined to include all posttranscri phonal and transcriptional mechanisms of RNA mediated inhibition of gene expression, such as those described in P. D. Zamore Science 296, 1265 (2002) which is incorporated by reference herein in its entirety. The discussion that follows focuses on the proposed mechanism of RNA interference mediated by short interfering RNA as is presently known, and is not meant to be limiting and is not an admission of prior art.

RNAi is a conserved biological response that is present in many, if not most, eukaryotic organisms. RNAi results in transcript silencing that is both systemic and heritable, permitting the consequences of altering gene expression to be examined throughout the development and life of an animal.

In the RNAi process, long double-stranded RNA molecules (dsRNA) can induce sequence-specific silencing of gene expression in primitive and multicellular organisms. These long dsRNAs are processed by a ribonuelease called Dicer into 21 to 23 nucleotide (nt) guide RNA duplexes termed short interfering RNA (siRNA). The siRNA is subsequently used by an RNA-induced silencing complex (RISC), a protein-RNA effector nuclease complex that uses siRNA as a template to recognize and cleave RNA targets with similar nucleotide sequences. The composition of RISC is not completely defined, but includes argonauts family proteins. The RISC unwinds siRNAs and associates stably with the (antisense) strand that is complementary to the target mRNA. Depending on the degree of homology between a siRNA and its target mRNA, siRNA-RISC complexes inhibit gene function by two distinct pathways. Most siRNAs pair imperfectly with their targets and silence gene expression by translationsl repression. This RNAi mechanism appears to operate most efficiently when multiple siRNA-bindlng sites are present in the 3′-untranslated region of the target mRNAs. In some other cases, siRNAs exhibit perfect sequence identity with the target mRNA and inhibit gene function by triggering mRNA degradation. The reduction in transcript level results in lowered levels of the target protein, resulting in phenotypic changes.

While siRNA has been shown to be effective for short-term gene inhibition in certain transformed mammalian cell lines, there may be drawbacks associated with its use in primary cell cultures or for stable transcript knockdown because their suppressive effects are by definition of limited duration. Short hairpin RNAs (skRNA), consisting of short, duplex structures, in contrast, to siRNAs have been proved as effective triggers of stable gene silencing in plants, in C. elegans, and in Drosophila. These synthetic forms of RNA may be expressed from pol II or pol III promoters and the hairpin structure is recognized and cleaved by Dicer to form siRNA that is subsequently taken up by RISC for silencing of the target gene.

In an embodiment, the compositions of this disclosure are able to reduce the level of expression of an Hsp, alternatively an eukaryotic Hsp, alternatively a mammalian Hsp. For example, the shRNAs of this disclosure may reduce the expression of a murine Hsp (e.g., Hsp25), a human Hsp (e.g., Hsp27), or both. In some embodiments, a nucleic acid molecule is able to reduce the expression of polypeptides produced from siRNA transcripts having the corresponding cDNA sequence set forth in SEQ ID NO:1 (5′-gcatggggaggggcggccctcaaacgggtcattgccattaatagagacctcaaacaccgcctgctaaaaatacccgactggaggagcat aaaagcgcagccgagcccagcgccccgcacttttctgagcagacgtccagagcagagtcagccagcatgaccgagcgccgcgtcccct tctcgctcctgcggggccccagctgggaccccttccgcgactggtacccgcatagccgcctcttcgaccaggc cttcggggctg ccccggctgc eggaggagtg gtcgcagtgg ttaggcggcagcagctggcc aggctacgtg cgccccctgc cccccgccgc catcgagagc cccgcagtggccgcgcccgc ctacagccgc gcgctcagcc ggcaactcag cageggggtc tcggagatccggcacactgc ggaccgctgg cgcgtgtccc tggatgtcaa ccacttcgcc ccggacgagctgacggrcaa gaccaaggar ggcgtgtgg agatcaccgg caagcacgag gagcggcaggacgagcatgg ctacatctcc cggtgcttca cgcggaaata cacgctgccc cccggtgtggaccccaccca agtttcctcc tccctgtccc ctgagggcac actgaccgtg gaggcccccatgcccaagct agccacgcag tccaacgaga tcaccatccc agtcaccttc gagtcgegggcccagcttgg gggcccagaa gctgcaaaat ccgatgagac tgccgccaag taaagccttagcccggatgc ccacccctgc tgccgccact ggctgtcct cccccgccac ctgtgtgttcttttgataca tttatcttct gtttttctca aataaagttc aaagcaacca cctgtcaaaaaaaaaaaaaaa aaaa-3′; NM 001540, which is hereby incorporated by reference).

In some embodiments, the compositions of this disclosure may comprise one nucleic acid, molecule that is able to reduce the expression of multiple Hsp. Alternatively, one nucleic acid molecule of the type described herein may exhibit cross, reactivity such that it Is able to reduce the expression of Hsp from differing species. In either embodiment, the single nucleic acid molecule may inhibit the expression of the differing Hsp to the same extent or to a differing extent. It is also contemplated that the compositions of this disclosure may also reduce the level of expression of one or more Hsp in non-mammalian systems.

The compositions of this disclosure comprise one or more nucleic acid molecules. In an embodiment, the nucleic acid molecule comprises a double stranded ribonucleic acid (dsRNA) molecule that inhibits the expression of a target gene wherein the dsRNA molecule comprises two strands of nucleotides wherein the first strand is substantially identical to the nucleotide sequence of SEQ ID NOs: 3, 5, 7, 9, or 11 and wherein the second strand is substantially complementary to the first strand. Herein substantially identical refers to greater than about 50% homology while substantially complementary refers to a complementarity sufficient to permit the annealing of the second strand to the first strand under biological conditions such as within the cytoplasm of a eukaryotic cell.

In an embodiment, the first snand is greater than about 55% identical, alternatively greater than about 60%, 65%, 70%, 75%, 80%, 90%, 95% identical to a complementary region of SEQ ID NO:1. The first strand may be of sufficient length such that it is processed by Dicer to produce an siRNA. Either strand may serve as a substrate for Dicer.

The length of each strand generally is from about 19 to about 25 nt in length (e.g., 19, 20, 21, 22, 23, 24, or 25 nucleotides). In some embodiments, the length of each strand is from about 19 to about 28 nucleotides In length. In one embodiment, the length of the sequence in the first strand is identical to the length of the sequence in the second strand and the dsRNA formed is blunt ended. In an alternative embodiment, the ends of the dsRNA formed has overhangs.

In an embodiment, an dsRNA for use in reducing the level of expression of a mammalian Hsp comprises a first strand which includes the RNA equivalent of the sequence 5′-AGCCCGAGCTGGGAACCATT-3′ (SEQ ID NO:2); in another embodiment the first strand includes the RNA equivalent of the sequence of 5′-CCGCAGAGCGTTTGAGTAT-3′ (SEQ ID NO:4). In an embodiment, a composition for use in the reduction of expression of a Hsp comprises a dsRNA having a first strand which includes the RNA equivalent of the sequence 5′ GCTCAATCCGAGAGAGAATA-3′(SEQ ID NO:6) and a second strand having a sequence complementary to the first strand. In an embodiment, the complementary first and second strands of the dsRNA molecule are the “stem” of a hairpin structure.

The two dsRNA strands can be joined by a binding moiety, which can form the “loop” in the hairpin structure of shRNA. In an embodiment the binding moiety comprises a polynucleotide linker which can vary in length. In some embodiments, the binding moiety can be 5, 6, 7, 8, 9, 10, 11, 12 or 13 nucleotides in length, alternatively the binding moiety is 9 nucleotides in length. A representative binding moiety is 5′-TTC AAG AGA-3′, but any suitable binding moiety that is compatible with, the formation of a dsRNA of the type disclosed herein, is contemplated. The two strands and binding moiety described herein may form a shRNA that can reduce the expression of one or more Hsp.

Nucleic acid molecules (e.g., dsRNA, shRNA) as described herein can be obtained using techniques known to one of ordinary skill, in the art. such as for example, recombinant nucleic acid technology; chemical synthesis, either as a single nucleic acid molecule or as a series of oligonucleotides; mutagenesis using common molecular cloning techniques (e.g., site-directed mutagenesis); and the polymerase chain reaction (PGR). General PGR techniques are described, for example in PGR Primer: A Laboratory Manual, Diefienbach & Dveksler, Eds., Cold Spring Harbor Laboratory Press, 1995 which is incorporated by reference herein in its entirety. Possible mutations include, without limitation, deletions, insertions, substitutions, and combinations thereof. Additionally, suitable molecular biology techniques may be employed for isolation of these molecules such as for example and without limitation restriction enzyme digestion and ligation.

As is known in the art, a nucleoside is a base-sugar combination. The base (or nucleobase) portion of the nucleoside is normally a heterocyclic base moiety. The two most common classes of such heterocyclic bases are purines and pyrimidines. Nucleotides are nucleosides that further include a phosphate group covalently linked to the sugar portion, of the nucleoside, for those nucleosides that include a pentofuranosyl sugar, the phosphate group can be linked to the 2′, 3′ or 5′ hydroxyl moiety of the sugar. In forming oligonucleotides, the phosphate groups covalently link adjacent nucleosides to one another to form a linear polymeric compound. The respective ends of this linear polymeric structure can be joined to form a circular structure by hybridization or by formation of a covalent bond. In addition, linear compounds may have internal nucleobase complementarity and may therefore fold in a manner as to produce a fully or partially double-stranded structure. Within the unmodified oligonucleotide structure, the phosphate groups are commonly referred to as forming the mtemueleoslde linkages of the oligonucleotide. The unmodified Internoeieoside linkage of RNA and DNA is a 3′ to 5′ phosphodiester linkage.

In the context of this disclosure, the term “unmodified oligonucleotide” refers generally to an oligomer or polymer of ribonucleic acid (RNA) or deoxyribonucleic acid (DNA). In some embodiments a nucleic acid molecule is an unmodified oligonucleotide. This term includes oligonucleotides composed of naturally occurring nucieobases, sugars and covalent internucleoside linkages. The term “oligonucleotide analog” refers to oligonucleotides that have one or more non-naturally occurring portions which function in a similar manner to oligonucleotides. Such non-naturally occurring oligonucleotides are often selected over naturally occurring forms because of desirable properties such as. for example, enhanced cellular uptake, enhanced affinity for other oligonucleotides or nucleic acid, targets and increased stability in the presence of nucleases. The term “oligonucleotide” can be used to refer to unmodified oligonucleotides or oligonucleotide analogs.

Specific examples of nucleic acid molecules include nucleic acid molecules containing modified, i.e., non-naturally occurring internucleoside linkages. Such non-naturally internucleoside linkages are often selected over naturally occurring forms because of desirable properties such as, for example, enhanced cellular uptake, enhanced affinity for other oligonucleotides or nucleic acid targets and increased stability in the presence of nucleases.

Nucleic acid molecules can have one or more modified internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides.

One suitable phosphorus-containing modified internucleoside linkage is the phosphorothioate internucleoside linkage. A number of other modified oligonucleotide backbones (internucleoside linkages) are known in the art and may be useful in the context of this invention.

Representative U.S. patents that teach, the preparation of phosphorus-containing internucleoside linkages include, but are not limited to, U.S. Pat Nos. 3,687,808; 4,469,863; 4,476,301; 5,023,243, 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,671,697 5,625,050, 5,489,677, and 5,602,240 each of which is herein incorporated by reference.

Modified oligonucleoside backbones (internucleoside linkages) that do not include a phosphorus atom therein have internucleoside linkages that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having amide backbones; and others, including those having mixed. N, O, S and CH₂ component parts.

Representative U.S. patents that teach the preparation of the above non-phosphorous-containing oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference.

Oligomeric compounds can also include oligonucleotide mimetics. The term mimetic as it is applied to oligonucleotides is intended to include oligomeric compounds wherein only the furanose ring or both the furanose ring and the internucleotide linkage are replaced with novel groups, replacement of only the furanose ring with for example a morpholino ring, is also referred, to in the art as being a sugar surrogate. The heterocyclic base moiety or a modified heterocyclic base moiety is maintained for hybridization with an appropriate target nucleic acid.

Oligonucleotide mimetics can include oligomeric compounds such as peptide nucleic acids (PNA) and cyclohexenyl nucleic acids (known as CeNA, see Wang et ah, J. Am, Chem. Soc, 2000, 122, 8595-8602) Representative U.S. patents that teach the preparation of oligonucleotide mimetlcs include, but are not limited to, U.S. Pat Nos. 5,539,082; 5,714,331; and 5,719,262, each of which is herein incorporated by reference. Another class of oligonucleotide mimetic is referred to as phosphonornonoester nucleic acid, and incorporates a phosphorus group in the backbone. This class of olignucieotide mimetic is reported to have useful physical and biological and pharmacological properties in the areas of inhibiting gene expression (antisense oligonucleotides, rihozymes, sense oligonucleotides and triplex-forming oligonucleotides), as probes for the detection of nucleic acids and as auxiliaries for use in molecular biology. Another oligonucleotide mimetic has been reported wherein the furanosyl ring has been replaced by a cyclobutyl moiety.

Nucleic acid molecules can also contain one or more modified or substituted, sugar moieties. The base moieties are maintained for hybridization with an appropriate nucleic acid target compound. Sugar modifications can impart nuclease stability, binding affinity or some other beneficial biological property to the oligomeric compounds.

Representative modified sugars include carhoeyclic or acyclic sugars, sugars having substituent groups at one or more of their 2′, 3′ or 4′ positions, sugars having substituents in place of one or more hydrogen atoms of the sugar, and sugars having a linkage between any two other atoms in the sugar. A large number of sugar modifications are known in the art, sugars modified at the 2′ position and those which have a bridge between any 2 atoms of the sugar (such that the sugar is bicyclic) are particularly useful in this invention. Examples of sugar modifications useful in this invention include, but are not limited to compounds comprising a sugar substituent group selected from: OH; F; O—, S—, or N-alkyl; or O-alkyl-O-alkyl, wherein the alkyl, alkenyl and alkynyl may be substituted or imsubstituted C₁ to C₁₀ alkyl or C₂ to C₁₀ alkenyl and alkynyl. Particularly suitable are: 2-methoxyethoxy (also known as 2′-O-methoxyethyl, 2MOE, or 2′-OCH₂CH₂OCH₃) 2′-O-methyl (2′-O—CH₃), 2′-fluoro (2′-F), or bicyclic sugar modified nucleosides having a bridging group connecting the 4′ carbon atom to the 2′ carbon atom wherein example bridge groups include —CH₂—O—, —(CH₂)₂—O— or —CH₂—N(R₃)—O wherein R₃ is H or C₁-C₁₂ alkyl.

One modification that imparts increased nuclease resistance and a very high binding affinity to nucleotides is the 2′-MOE side chain (Baker et al., J. Biol. Chem., 1997, 272, 11944-12000). One of the immediate advantages of the 2′-MOE substitution is the improvement in binding affinity, which is greater than many similar 2′ modifications such as O-methyl, O-propyl, and O-aminopropyl. Oligonucleotides having the 2′-MOE substituent also have been shown to be antisense inhibitors of gene expression with promising features for in vivo use (Martin, P., Helv. Chirm Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16,917-926).

2′-Sugar substituent groups may be in the arabino (up) position or ribo (down) position. One 2′-arabino modification is 2′-F. Similar modifications can also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimeti.es such as cyclobntyl moieties in place of the pentofnranosyi sugar. Representative U.S. patents that teach me preparation of such modified sugar structures include, but are not limited to, U.S. Pat. Nos. 4,981,957; 5,118,800; 5,319,080; 5,359,044; 5,393,878; 5,446,137, 5,466,786; 5,514,785; 5,519,134; 5,567,811; 5,576.427; 5,591,722; 5,597,909; 5,610,300; 5,627,053; 5,639,873; 5,646,265; 5,658,873; 5,670,633; 5,792,747; and 5,700,920, each, of which is herein incorporated by reference in its entirety.

Representative sugar substUucnts groups are disclosed in U.S. Pat. No. 6,172,209 entitled “Capped 2′-Oxyethoxy Oligonucleotides,” hereby incorporated by reference in lis entirety.

Representative cyclic sugar substituent groups are disclosed in U.S. Pat. No. 6,271,358 entitled “RNA Targeted 2′-Oligemeric compounds that are Conformationally Preorganized.” hereby incorporated by reference in its entirety.

Representative guanklino substituent groups are disclosed in U.S. Pat. No. 6,593,466 entitled “Functionalized Oligomers,” hereby incorporated by reference in its entirety.

Representative acetamido substituent groups are disclosed in U.S. Pat. Net. 6,147,200 which is herebv incorporated by reference in its entirety.

Nucleic acid molecules can also contain one or more nueleoba.se (often referred to in the art simply as “base”) modifications or substitutions which are structurally distinguishable from, yet functionally interchangeable with, naturally occurring or synthetic unmodified nucleobases. Such nucleobase modifications can impart nuclease stability, binding affinity or some other beneficial biological property to the oiigomerie compounds. As used herein, “unmodified” or “natural” nucleobases include the purine bases adenine (A) and guanine (G), and the pyrimidine bases thymine (T), cytosine (G) and uracil (U). Modified nucleobases also referred to herein as heterocyclic base moieties include other synthetic and natural nucleobases, many examples of which such as 5-methylcytosine (5-me-C), 5-hydroxymethyl cytosine, 7-deazaguanine and 7-deazaadenine among others.

Heterocyclic base moieties can also include those in which the purine or pyrimidine base is replaced with other heteroeyeles, for example 7-deaza-adenine, 7-deazaguauesine, 2-aminopyridine and 2-pyridone. Some nucleobases include those disclosed in U.S. Pat. No. 3,687,808, those disclosed in The Concise Encyclopedia Of Polymer Science And Engineering, pages 858-859, Krosehwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucieobases are particularly useful for increasing the binding affinity of the ollgonveric compounds of the invention. These include 5-subsiituted pyrimidines, 6-azapyrimidines and N-2, N-6 and O-6 substituted purines, including 2 aminopropyladenine, 5-propynyluracil and 5-propynylcytosine.

Additional modifications to nucleic acid molecules are disclosed in U.S. Patent Publication 2009/0221685, which is hereby incorporated by reference.

The nucleic acid molecules disclosed herein may be introduced to a cell directly using techniques such as for example encapsulation in a nanoparticie or a liposome; electroporation; calcium phosphate precipitation and the like. In some embodiments, one or more nucleic acid molecules may be introduced to a cell as an element of a vector and thus comprise a DNA vector-based shRNA. Hereinafter, for simplicity the discussion will focus on compositions comprising shRNA although other compositions of the type described previously herein are also contemplated.

Vectors, including expression vectors, suitable for use in the present disclosure are commercially available and/or produced by recombinant DNA technology methods routine in the art. A vector containing a shRNA of this disclosure may have elements necessary for expression operably linked to such a molecule, and further can include sequences such as those encoding a selectable marker (e.g., a sequence encoding antibiotic resistance), and/or those that can be used in purification of a polypeptide (e.g., a His tag). Vectors suitable for use In this disclosure can integrate into the cellular genome or exist extraeliromosomally (e.g., an autonomous replicating plasmid with an origin of replication).

In an embodiment, the vector is an expression vector and comprises additional elements that are useful for the expression of the nucleic acid molecules of this disclosure. Elements useful for expression include nucleic acid sequences that direct and regulate expression of nucleic acid coding sequences. One example of an element useful for expression is a promoter sequence. Examples of promoters suitable for use include the mouse U6 RNA promoters, synthetic human H1RNA promoters, SV40, CMV, RSV, RNA polymerase II, RNA polymerase II promoters, derivatives thereof, or combinations thereof. Elements useful for expression also can include ribosome-bindiag sites, introns, enhancer sequences, response elements, or inducible-elements that modulate expression of a nucleic acid. Elements necessary for expression can be of bacterial, yeast, insect, mammalian, or viral origin and the vectors may contain a combination ofelements from different origins. Elements necessary for expression are known to one of ordinary skill in the art and are described, for example, in Goeddei, 1990, Gene Expression Technology; Methods in Enzyrnology, 185, Academic Press, San Diego, Calif., the relevant portions of which are incorporated by reference herein. As used herein, operably linked means that a promoter and/or other regulatory eiementfs) are positioned in a vector relative to the shRNA in such a way as to direct or regulate expression of the molecule. A shRNA can he operably-iinked to regulatory sequences in a sense or antisense orientation. In addition, expression can refer to the transcription of sense mRNA and may also refer to the production of protein.

In an embodiment, the shRNAs of the present disclosure are elements of a retroviral vector. A retroviral vector refers to an artificial DNA construct derived from a retrovirus that may be used to insert sequences into an organism's chromosomes. Adenovirus and a number of retroviruses such as lentivims and murine stem cell virus (MSCV) are a few of the commonly used, retroviral delivery systems. Adenovirus utilizes receptor-mediated infection and does not integrate into the genome for stable silencing experiments, while MSCV cannot integrate into non-dividing cell lines such as neurons, etc. A lentiviral vector is a subclass of retroviral vectors that have the ability to integrate into the genome of non-dividing as well as dividing- cells, Lentiviral. vectors are known in the art, and are disclosed, for example, in the following publications, which are incorporated herein by reference: Evans J. T. et al. Hum. Gene Ther, 1999; 10:1479-1489; Case S. S., Price, M. A., Jordan C. T. et al. Froc. Natl. Acad. Sci. USA 1999; 96:2988-2993; Uchida N., Sutton R. E., Friera, A. M. et al. Proc. Natl. Acad. Sci. USA 1998; 95:11939-11944; Miyoshi H, Smith K A, Mosier D. E et al. Science 1999; 283:682-686; Sutton R. E., Wu H. T., Rigg R. et al. J. Virol, 1998; 72:5781-5788. The lentiviral vector systems display a broad tropism and non-receptor mediated delivery. Furthermore, lentiviral vector systems have the ability to integrate into the genome for stable gene silencing, without requiring a mitotic event for integration into the genome; thus, extending Its use to both dividing and nondividing cell lines. The lentiviral vector system. Is also not known to elicit immune responses minimizing concerns of off-target effects and use in in vivo applications.

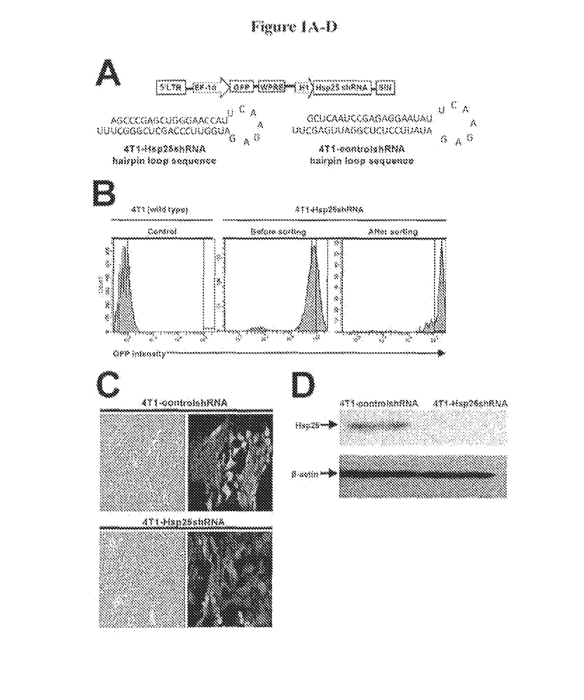

In an embodiment, the shRNAs of the present disclosure are elements of a lentrviral vector. A vector diagram representing an embodiment of a vector suitable for use in this disclosure is shown in FIG. 1. Referring to FIG. 1, features of a typical vector for use in the present disclosure include a promoter such as the elongation factor alpha 1 promoter (EF-1a) disposed upstream of at least one positive selection marker such as the green fluorescent protein (GFP); and one or more regulatory elements such as for example and without limitation the woodchuck hepatitis post-transeriptionai regulatory element (WPRE); and at least one nucleic acid molecule sequence tor the reduction of Hsp expression (e.g., an shRNA having a first strand comprising SEQ ID NO:4, a complementary second strand and a binding moiety) whose expression may be driven by an upstream, polymerase III promoter, human 1 (H1). A regulatory element refers to a genetic element designed to enhance expression of the gene of interest. In one embodiment, the lentrviral vector contains an H1-RNA promoter that is operably linked to a nucleic acid sequence encoding a nucleic acid molecule containing at least one of the sequences previously disclosed herein. Thus, the H1 promoter Initiates the transcription of the nucleic acid molecule and allows for the constitutive expression of the nucleic acid molecule, in another embodiment, the nucleic acid molecule is operahly linked to a regulatable promoter that provides inducible expression of the nucleic acid molecule. Such inducible promoters and methods of using same are known to one of ordinary skill in the art. In an embodiment, the vector is a lentiviral vector and the markers, genes and other elements of vector may be flanked by an intact retroviral 5′ long terminal repeat (LTR) and 3′ self inactivating (SIN), Such flanking sequences are known, to one of ordinary skill in the art.

The types of elements that may be Included in the construct are not limited in any way and will be chosen by the skilled practitioner to achieve a particular result For example, a signal that facilitates nuclear entry of the viral genome in the target cell may be included in the construct. It is to be understood that minor modifications of the vector as disclosed herein may be made without significantly altering the utility of the vector. As such, the vector diagram is not intended to be limiting and is illustrative of one embodiment of a family of vectors. For simplicity hereinafter the family of vectors comprising at least one shRNA as disclosed herein will be referred to as the beat shock protein reduction vector (HRV). In an embodiment, the HRV comprises a lentrviral vector such as for example the LentiGFP Vector commercially available from Lentigen Corp. of Baltimore, Md., the Block-iT Lentiviros Vector commercially available from lirvitrogen of Carlsbad, Calif., and the pSIF1-H1 shRNA Vector commercially available from System Biosciences of Mountain View, Calif. and a shRNA of this disclosure.

In an embodiment, the HRV comprises one or more expression cassettes wherein the expression cassette comprises a promoter operably-linked to an isolated nucleic acid sequence encoding a first segment, a second segment located immediately 3′ of the first segment, and a third segment located immediately 3′ of the second segment wherein the first and third segments are from about 19 to about 28 nucleotides in length and wherein the first segment is substantially identical to any of SEQ ID NOs 2-11 and wherein the sequence of the third segment is the complement of the first segment. In an embodiment, the isolated nucleated acid sequence expressed from the HRV functions as a shRNA that inhibits the expression of one or more Hsp.

The HRV may be delivered to cells in any way that allows the virus to infect the cell. In an embodiment, the HRV is introduced into a packaging cell line. The packaging cell line provides the viral proteins that are required in trans for the packaging of the viral genomic RNA into viral particles. The packaging cell line may be any cell line that is capable of expressing retroviral proteins. The HRV may then be purified from the packaging cells, titered and diluted to the desired concentration. In one embodiment, the infected cells may be used with or without further processing. In another embodiment, the infected cells may he used to infect an organism,

In an embodiment, the HRV is introduced to a cell or cell line. In another embodiment, the HRV may be introduced to a non-human animal as a genetically modified cell and maintained by the non-human animal in vivo for some period of time. For example, cells may be isolated from the non-human animal and the HRV introduced into cells using any number of in vitro techniques as have been described previously herein (e.g, electroporation, calcium phosphate precipitation, etc.). The isolated cells now carrying the HRV may be reintroduced to the non-human animal and result In the reduced expression of one or more Hsps for some period of time. In other embodiments, similar methodologies may be employed for treating a human having an undesired condition.

In an embodiment, cells, tissue, or an organism having been infected with air HRV as disclosed herein may experience a reduced level of Hsp expression when compared to an otherwise similar cell or organism lacking an HRV. For example, cells expressing a Hsp when infected with an HRV comprising any of SEQ ID NOS 2-11 may experience a reduction in the level of Hsp expression.

The Hsp expression level is a cell or organism comprising an HRV may be reduced by an amount of equal to or greater than about 60%, alternatively greater than about 70, 75, or 80% when compared to an otherwise identical cell or organism, in die absence of an HRV. Methods, for determining the reduction in the Hsp expression level may comprise, assays for the mRNA transcript; assays for the translated product, or combinations thereof. Nucleic acid molecules (e.g., mRNA transcript) and polypeptides (e.g., Hsp) can he detected using a number of different methods well known to one of ordinary skill in the art. Methods for detecting nucleic acid molecules include, for example, PGR and nucleic acid hybridizations (e.g., Southern blot, Northern blot, or in situ hybridizations).

The shRNAs of the present disclosure can be used to reduce the expression of Hsp in a number of cell types or tissue types. As such the shRNAs may be introduced to any cell type or tissue experiencing an undesirable condition for which reduction of the expression of Hsp may ameliorate said condition. For example, the shRNAs of the present disclosure can be used to reduce the expression of Hsp in cancer cells. As used herein, “cancer cells” refer to cells that grow uncontrollably and/or abnormally, and can be, for example, epithelial carcinomas. Epithelial, carcinomas include, for example, head and neck cancer cells, breast cancer cells, prostate cancer cells, and colon cancer cells. The shRNAs of the present disclosure may be administered so as to result in an inhibition of the proliferation of cancer cells, Proliferation of cancer cells as used herein refers to an increase in the number of cancer cells (in vitro or in vivo) over a given period of time (e.g., hours, days, weeks, or months). It is noted that the number of cancer cells is not static and reflects both the number of cells undergoing cell division and die number of cells dying (e.g., by apoptosis). An Inhibition of the proliferation of cancer cells can be defined as a decrease in the rate of increase in cancer cell number, a complete loss of cancer cells, or any variation there between. With respect to tumors, a decrease in the siixe of a tumor can be an indication of an inhibition of proliferation. The administration of one or more compositions comprising an shRNA of the type described herein to an organism having a cell proliferation disorder evinced by tumor growth, may result in an inhibition of rumor growth of from about 10% to about 90%, alternatively from about 30% to about 90%, alternatively greater than about 75% when compared to the tumor cell growth observed in the absence of the HRV. Herein the tumor cell growth refers to cell proliferation or increase in tumor mass and may be measured by techniques known to one of ordinary skill in the art such as for example magnetic resonance imaging, electronic caliper, mammogram.

Further, the shRNAs of the present disclosure may result in the cancer having a reduced metastatic potential. Metastasis refers to the spread of cancerous cells from its primary site to other sites in the body. Thus, the shRNAs of this disclosure when introduced and expressed in cancer cells having a metastatic potential may reduce the ability of the cancerous cells to spread from the primary site when compared to the metastatic potential of cells not expressing the shRNAs of this disclosure. The administration of one or more compositions comprising an shRNA of the type described herein to an organism having a cell proliferation disorder evinced by tumor growth with the potential to metastasize may result in reduction in the metastatic potential of from about 10% to about 95%, alternatively from about 30% to about 70%, alternatively equal to or greater than about 75% when compared to the tumor cell growth observed in the absence of the HRV. Herein metastatic potential refers to the ability of the tumor to grow at one more distal sites and may be measured, by techniques known to one of ordinary skill in the art such as for example cell migration assays.

In an embodiment, the compositions comprising shRNAs of the type described herein may be used in conjunction with other therapeutic methods to effect the treatment of an undesirable condition. For example, the shRNAs of this disclosure may be used in conjunction with other gene silencing therapies, chemotherapeutie regimes, radiation therapies, hypothermia, and the like.

In an embodiment, the shRNAs of this disclosure may he a component in a pharmaceutical composition wherein the composition is to be administered to an organism experiencing an undesired condition and act as a therapeutic agent. The pharmaceutical composition (PC) may be formulated to be compatible with its Intended route of administration. For example, the organism may have one or more tumor loads and the PC may he Introduced via direct injection. Additionally, examples of routes of administration include parenteral, (e.g., intravenous, intradermal, subcutaneous); oral (e.g., ingestion or inhalation); transdermal (e.g., topical); transmucosal; and rectal administration. In an embodiment, the shRNAs of the present disclosure either alone or as a component of a vector (i.e. HRV) can be incorporated into pharmaceutical compositions suitable for administration. Such compositions typically comprise the shRNAs, and a pharmaceutlcaliy acceptable carrier or exeipient. As used herein, “pharmaceutically acceptable carrier” is intended to include any and all solvents, dispersion media, coatings, antibacterial and anti-fungal agents, isotonic and absorption delaying agents, and the like, compatible, with pharmaceutical administration. The use of such media and agents for pharmaceutically active substances is well known in the art.

In an embodiment, a composition for use in the treatment of an undesirable condition comprises administration of a tumor targeting Hsp reduction system (TTHRS). The TTHRS may comprise one or more of the Hsp compositions previously described herein, one or more delivery nanopariicies, and one or more targeting moieties. In an embodiment, the TTHRS is capable of delivering the Hsp reducing compositions of this disclosure to tumor cells wherever they may occur in the body. For example, the TTHRS may be capable of delivering the compositions of this disclosure to both primary and metastatic disease.

In an embodiment, the TTHRS comprises a delivery system for the transport of one or more shRNAs and optional components in an organism. Delivery systems may include the use of any materials compatible with the compositions of this disclosure and suitable for use in an organism. In an embodiment, the delivery system comprises a nanoparticle, alternatively a liposome. Herein nanoparticle refers to a material wherein at least one dimension is less than about 100 nm in stee while liposome refers to abilayer lipid, liposomes generally have systemic applications as they exhibit extended circulation lifetimes following intravenous (i.v.) injection, can accumulate preferentially in various tissues and organs or tumors due to the enhanced vascular permeability in such regions, and can be designed to escape the lyosomic pathway of endoeylosis by disruption of endosomal membranes. Liposomes generieally comprise an enclosed lipid droplet having a core, typically an aqueous core, containing the compound. The liposomes or liposome precursors may be prepared using any means known to one of ordinary skill in the art. An example of liposomes suitable for use in this disclosure are the DOTAP series of cationie lipids which are substituted N-(1-(2,3-dioleoyloxy)propyl)-N,N,N-trimethylanimonium chloride compounds commercially available from Avanti Polar Lipids. In certain embodiments, the Hsp reducing compositions of this disclosure are chemically conjugated to a lipid component of the liposome. In other embodiments, the Hsp reducing compositions of this disclosure are contained within the aqueous compartment inside the liposome.

Additionally disclosed herein are articles of manufacture (e.g., kits) that contain one or more shRNAs, one or more vectors that encode a shRNA of the present disclosure. Such compositions may be formulated for. administration and may be packaged appropriately for the intended route of administration as described previously herein. For example, a shRNA or a vector comprising a shRNA of the present disclosure can be contained within a pharmaceutically acceptable carrier or exciplent.

In an embodiment, a kit comprising a shRNA of the present disclosure also can include additional reagents (e.g., buffers, co-factors, or enzymes). Pharmaceutical compositions as described herein further can include instructions for administering the composition to an individual. The kit also can contain a control sample or a series of control samples that can be assayed and compared to the biological sample. Each component of the kit is usually enclosed within an individual container and all of the various containers are within a single package.

The nucleic acid molecules may be administered to a subject alone or in the form of a pharmaceutical composition for the treatment of a condition or disease, Pharmaceutical compositions may be formulated in conventional manner using one or more physiologically acceptable carriers, diluents, excipients or auxiliaries which facilitate processing of the proteins into preparations which can be used pharmaceutically. Proper formulation is dependent upon the route of administration chosen.

For topical, administration the nucleic acids may be formulated as solutions, gels, ointments, creams, suspensions, etc, as are well-known in the art. Systemic formulations include those designed for administration by injection, e.g. subcutaneous, intravenous, intramuscular, intrathecal or intraperitoneal injection, as well as those designed for transdermal, transmucosal, inhalation, oral or pulmonary administration. For injection, the nucleic acids of the invention may be formulated in aqueous solutions, preferably in physiologically compatible buffers such as Hanks' solution. Ringer's solution, or physiological saline buffer. The solution may contain formulatory agents such as suspending, stabilizing and/or dispersing agents. Alternatively, the nucleic acid molecules may be in powder form for constitution with a suitable vehicle, e.g., sterile pyrogen-free water, before use. For transmucosal administration, penetrants appropriate to the barrier to be permeated are used in the formulation. Such penetrants are generally known in the art. For oral administration, the nucleic acids can be readily formulated by combining the molecules with pharmaceuticaliy acceptable carriers well known in the art. Such carriers enable the nucleic acids of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, e.g. lactose, sucrose, mannitoi and sorbitol; cellulose preparations such as maize starch, wheat starch, rice starch, potato starch, gelatin, gum tragacanth, methyl cellulose, hydroxypropylmetbyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginlc acid or a salt thereof such as sodium alginate. If desired, solid dosage forms may be sugar-coated or enteric-coated using standard techniques. For oral liquid preparations such as, for example, suspensions, elixirs and solutions, suitable carriers, excipients or diluents include water, glycols, oils, alcohols, etc. Additionally, flavoring agents, preservatives, coloring agents and the like may be added. For buccal administration, the molecules may take the form of tablets, lozenges, etc. formulated in conventional manner. For administration by inhalation, the molecules for use according to the present invention are conveniently delivered in the form of an aerosol spray from pressurized packs or a nebulizer, with the use of a suitable propellant e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator may be formulated containing a powder mix of the nucleic acids and a suitable powder base such as lactose or starch. The nucleic acid molecules may also be formulated in rectal or vaginal compositions such as suppositories or retention enemas, e.g., containing conventional suppository bases such as cocoa butter or other glycerides.

In addition to the formulations described previously, the molecules may also be formulated as a depot preparation. Such long acting formulations may be administered by implantation (for example subcotaneously or intramuscularly) or by intramuscular injection. Thus, for example, the molecules may be formulated with suitable polymeric or hydrophobic materials (for example as an emulsion in an acceptable oil) or ion exchange resins, or as sparingly soluble derivatives, for example, as a sparingly soluble salt.

Alternatively, otter pharmaceutical delivery systems may be employed. Liposomes and emulsions are well-known examples of delivery vehicles that may be used to deliver nucleic acids of the inventlon.

A nucleic acid molecule may be administered in combination with a carrier or lipid to increase cellular uptake. For example, the oligonucleotide may be administered in combination with a cationic lipid. Examples of cationic lipids include, but are not limited to, lipofectin, DOTMA, DOPE, and DOTAP. The publication of WO0071096, which is specifically incorporated by reference, describes different formulations, such as a DOTAP:cholesterol or cholesterol derivative formulation that can effectively be used for gene therapy. Other disclosures also discuss different lipid or liposomal formulations including nanopariides and methods of administration; these include, but are not limited to, U.S. Patent Publication 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

The nucleic acids may also be administered in combination with a cationic amine such as poly (L-lyslne), Nucleic acids may also be conjugated to a chemical moiety, such as transferrin and eholesteryls. In addition, oligonucleotides may be targeted to certain organelles by linking specific chemical groups to the oligonucleotide, For example, linking the oligonucleotide to a suitable array of mannose residues will target the oligonucleotide to the liver.

Additionally, the molecules may be delivered using a sustained-release system, such as semipermeable matrices of solid polymers containing the therapeutic agent. Various of sustained-release materials have been established and are well known by those skilled in the art. Sustained-release capsules may, depending on their chemical nature, release the molecules for a few weeks up to over 100 days. Depending on the chemical nature and the biological stability of the chimeric molecules, additional strategies for molecule -stabilization may be employed.

Nucleic acids may be included in any of the above-described formulations as the free acids or bases or as pharrnaceoueally acceptable salts. Pharmaceutically acceptable salts are those salts that substantially retain the biologic activity of the free bases and which are prepared by reaction with inorganic acids, Pharmaceutical sails tend to be more soluble in aqueous and other protic solvents than ate the corresponding free base forms.

Pharmaceutical compositions of the present invention comprise an effective amount of one or more synthetic nucleic acid molecules dissolved or dispersed in a pharmaceutically acceptable carrier. The phrases “pharmaceutical, or pharmacologically acceptable” refers to molecular entities and compositions that do not produce an adverse, allergic or other untoward reaction when administered to an animal, such as, for example, a human, as appropriate. The preparation of an pharmaceutical composition that contains at least one chimeric polypeptide or additional active ingredient will be known to those of skill in the art in light of the present disclosure, as exemplified, by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption, delaying agents, salts, preservatives, drugs, drug stabilizers, gels, binders, excipients, disintegration agents, lubricants, sweetening, agents, flavoring agents, dyes, such like materials and combinations thereof, as would be known to one of ordinary skill in the art (see, for example, Remington's Pharmaceutical Sciences, 18th Rd. Mack Printing Company, 1990, pp. 1289-1329, incorporated herein by reference). Except insofar as any conventional carrier is incompatible with the active ingredient, its use in the therapeutic or pharmaceutical compositions is contemplated.

The molecules may comprise different types of carriers depending on whether it is to be administered in solid, liquid or aerosol form, and whether it need to be sterile for such routes of administration as injection.

The nucleic acid, molecules or compositions containing nucleic acid molecules can be administered intravenously, intradermally, intraarterially, intraperitoneally, intralesionally, intracranially, intraarticularly, intraprosiaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraoculaxally, orally, topically, locally, inhalation (e.g. aerosol inhalation), injection, infusion, continuous infusion, localized perfusion bathing target cells directly, via a catheter, via a lavage, In cremes, in lipid compositions (e.g., liposomes), or by other method or any combination of the forgoing as would be known to one of ordinary skill in the art (see, for example, Remington's Pharmaceutical Sciences, 18th Ed. Mack, Printing Company, 1990, incorporated herein by reference).

The actual dosage amount of a composition that Is administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 miliigram/kg/body weight, about 1.0 milligram/kg/body weight, about 50 milligram/kg/body weight, about 100 milligram/kg/body weight, about 200 milligram/kg/body weight, about 350 milligram/kg/body weight, about 500 milligram/kg/body weighty to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/hody weight, etc, can be administered, based on the numbers described above.

In any ease, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The molecules may be formulated into a composition in a free base, neutral or salt form. Pharmaceatically acceptable salts, include the acid addition salts, e.g., those formed with the free amino groups of a proteioaceous composition, or which are formed with inorganic acids such as for example, hydrochloric or phosphoric acids, or such organic acids as acetic, oxalic, tartaric or mandelic acid. Salts formed with the free carboxyi groups can also be derived from inorganic bases such as for example, sodium, potassium, ammonium, calcium or ferric hydroxides; or such organic bases as isopropyiamine, trlmethyiamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods, in many cases, it will be preferable to include isotonic agents, such as, for example, sugars, sodium chloride or combinations thereof.

In other embodiments, one may use eye drops, nasal solutions or sprays, aerosols or inhalants in the present invention, Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions, usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments, the molecules are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc; or combinations thereof the foregoing. When the dosage unit form is a capsule, it may contain, in addition to materials of the above type, carriers, such as a liquid carrier. Various other materials may be present as coatings or to otherwise modify the physical form of the dosage unit. For instance, tablets, pills, or capsules may be coated with shellac, sugar or both.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg nucleic acid.

The molecules of the invention will generally be used in an amount effective to achieve the intended purpose. For use to treat or prevent a disease condition, the molecules of the invention, or pharmaceutical compositions thereof, are administered or applied in a therapeutically effective amount. A therapeutically effective amount is an amount effective to ameliorate or prevent the symptoms (such as tumor growth), or prolong the survival of, the patient being treated. Determination of a therapeutically effective amount is well within the capabilities of those skilled in the art, especially in light of the detailed disclosure provided herein.

For systemic administration, a therapeutically effective dose can he estimated initially from in vitro assays. For example, a dose can be formulated in animal models to achieve a circulating concentration ranee that includes the IC₅₀ as determined in cell culture. Such information can be used, to more accurately determine useful doses in humans.

Initial dosages can also he estimated from in vivo data, e.g., animal models, using techniques that are well known in the art. One having ordinary skill in the art could readily optimize administration to humans based ou animal data.

Dosage amount and interval may be adjusted individually to provide plasma levels of the molecules which are sufficient to maintain therapeutic effect. Usual patient dosages for administration by injection range from about 0.1 to 5 mg/kg/day, preferably from about 0.5 to 1 mg/kg/day. Therapeutically effective serum levels may be achieved by administering multiple doses each day.

In cases of local administration or selective uptake, the effective local concentration of the proteins may not be related to plasma concentration. One having skill in the art will be able to optimize therapeutically effective local dosages without undue experimentation.

The amount of molecules administered will, of course, be dependent on the subject being treated, on the subject's weight, the severity of the affliction, the manner of administration and the judgment of the prescribing physician.

The therapy may be repeated intermittently while symptoms detectable or even when they are not detectable. The therapy may be provided alone or in combination with other drugs or treatment (Including surgery).

### EXAMPLES

The invention having been generally described, the following examples are given as particular embodiments of the invention and to demonstrate the practice and advantages thereof. It is understood that the examples are given by way of illustration and are not intended to limit the specification of the claims to follow in any manner.

### Example1

### Hsp25shRNA inhibits Tumors, Materials and Methods

Cells and Culture Conditions

4T1 is a highly metastatic breast cancer cell line derived from a spontaneously arising BALB/c mammary tumor, BNL 1MEA.7R.1 (BNL) is a mouse transformed hepatocellular carcinoma (HCC) cell line derived from BALB/c mice. Both cells were purchased from American Type Cell Culture (ATCC; Rockville, Md.). 4T1 cells were maintained in monolayer cultures in DMEM (Cellgro, Los Angeles, Calif.) supplemented with 10% fetal bovine serum (FBS) and antibiotics/antimycoties (Invitrogen Life Technologies, Carlsbad, Calif.), Cells were maintained at 37° C. humidified atmosphere with 5% CO₂. BNL cells were maintained in Dulbeeco's Modified Eagle Medium (Sigma Chemicals, St. Louis, Mo.), supplemented with 10% heat-inactivated FBS, antibiotics and antimycostics (Gibco Bill/Life Technologies, Inc., Gaithersburg, Md.) in a humidified atmosphere of 5% CO₂ at 37° C.

Preparation of Small Hairpin RNA Mouse Hsp25 by Lentivirus Gene Transfer Vector

A HIV derived three plasmid system was kindly provided by Dr. Trono (Department of Microbiology and Molecular Medicine, University of Geneva, Switzerland). The plasrnid pLVTHM was digested with Mlu I and Cla I and ligated to an oligonucleotide pair containing Hsp25shRNA or controlshRNA carrying Mlu I and Cla I restriction overhangs and transformed into Max Stbl2 competent cells. Positive clones were identified by digesting the control pLVTHM vector and the vector containing Hsp25shRNA inserts using Mln I and Xba I enzymes, and confirmed by DNA sequencing, Lentivirus transfection was carried out according to the standard protocol (21). Briefly, cells were plated into six-well plates (3×10⁴ cells/well) and 1-ml concentrated, high titer virus (5×10⁸) was directly added to the cells. Polybrene was then added at a final concentration of 8 μg/ml and incubated for an additional 5 days in a 37° C. incubator. Transfection efficiency was determined by fluorescence microscopy and highly expressing cells were isolated using flow cytometry cell sorting.

Animals and Tumor Challenge

Female BALB/c (H2^{d}) wild type mice and female BALB/c nude mice (6-8 weeks old) were purchased from Charles River Laboratories (Wilmington, Mass.). Female C57BL/6 (H2^{b}) mice (6-8 weeks old) wore purchased from Jackson Labs (Bar Harbor, Mass.). All animals were housed under pathogen-free conditions in laminar flow isolation units in the Scott & White Hospital's vivarium under alternate dark and light cycles. Animals were maintained on food and-water ad libitum. For tumor challenge experiments, mice were either injected with 10⁴ 4T1 cells (suspended 0.2 ml PBS) into the lower right mammary gland, or with 10⁶ BNL tumor cells (suspended 0.2 ml PBS) into the right flank. The tumor volume was measured at regular intervals using an electronic caliper or non-invasiveiy using the Maestro™ in vivo imaging system (CRI, Woburn, Mass.). All animals were treated humanely and in accordance with the guidelines of the Committee on the Care and Use of Laboratory Animals of the Institute of Animal Resources, National Research Council and institutional Animal Care and Use Committee (IACUC) of Scott & White Hospital.

Live Animal Imaging

Live animal imaging was achieved by measuring the spectral fluorescence images captured using the Maestro™ in vivo imaging system (CRI). An excitation band pass filter from 445 to 490 nm and an emission filter over 515 nm were used. The tunable filter was automatically spaced in 10 nm increments from 500-720 nm while the camera captured fluorescence images at each wavelength with constant exposure, RGB (red-green-blne) color fluorescence images were synthesized from the spectral cube by mapping the spectral data into those color channels. All the fluorescence images obtained as RGB images were derived from the spectral; datasets. Spectral unmixing was performed to segregate skin and hair auto fluorescence and toi measure the true GFP signal.

Production of Bone Marrow-Derived Macrophages (BMDM) and In Vitro Cross-Presentation Assay

Femurs and tibias from female BALB/c (H2^{d}) mice or C57BL/6 (H2^{b}) mice were excised and flushed with ice-cold sterile DMEM (Cellgro) containing 10% FCS and antibiotics/antimycotics (Invitrogen Life Technologies), termed complete media. Bone marrow cells were treated with Red Blood Cell Lysis Buffer according to the manufacturers instructions (eBioscience, San Diego, Calif.) and incubated in complete media supplemented with 10 ng/ml M-CSF (R&D Systems, Minneapolis, Minn.). After 3 days incubation, an additional 10 ng/ml M-CSF was added to the culture media. On day 7, bone marrow-derived macrophages (BMDM) were seeded at 10⁴ cell, per well in 96-well plates and transfeeted with either Hsp25-siRHA or control-siRHA for 48 h. Control-siRNA is a non-targeting 20-25 nt siRNA designed as a negative control, with sequences that do not target any gene product nor has any significant sequence similarity to mouse, rat, or human gene sequences, and has been tested in cell-based screens and proved to have no significant effect on cell proliferation, viability, or morphology, according to the manufacturer (Amhlon, Austin, Tex.). BMDM were then pulsed with 100 ng/ml OVA peptide (S8L) or 100 ng/ml control peptide (PB1; a synthetic peptide purchased from New England Biolabs, Ipswich, Mass.) for 2 h and returned to a 37° C. incubator. BMDM were later washed to remove excess peptide and fixed with paraformaldehyde: for 10 min at room temperature. Peptide-specific T cell hybridoma (B3Z) was added to the fixed BMDM at 37° C. for 24 h, and the culture supernatant was recovered and the concentration of IFN-γ measured by classical sandwich ELISA.

In Vivo Antibody Depletion Assay

The in vivo depletion of CD4 T cells (using anti-CD4; L3T4 antibodies), CD8 T cells (using anti-CD8; Ly-2 antibodies) and NK cells (using anti-NK; 5E6 antibodies) was accomplished by i.p. injection of 100 μg antibody/mice once a week. All the antibodies were purchased from BD Bioscience (Franklin Lakes, NJ). The injection of antibodies started 4 days before injection of tumor cells and continued till the end of the experiment. In vivo depletion of specific cell subsets was confirmed by flow cytometric analysis of splenocyies one day before tumor challenge. Animals treated with isotype control were used as a negative control for antibody depletion.

Isolation of CD8⁺ and CD8⁻ T cells and In Vivo Adoptive Transfer Assay

Reactive CD8⁺ T cells were Isolated from the spleen of 4T1-Hsp25shRNA cell-bearing mice using the CD8⁺ T cell negative-selection kit according to manufacturers instructions (Milteny Biotec, Auburn, Calif.). Non-CD8⁺ T cells (containing CD4⁺ T cells, B cells, NR. cells, granulocytes and monocytes) were referred herein as CD8− T cells, and were isolated by depleting CD8⁺ T cells from die spleen of 4T1-Hsp25shRNA cell-bearing mice using the CD8⁺ T cell positive-selection kit according to manufacturers. instructions (Milteny Biotec). Adoptive transfer was achieved by the injection of 4T1-controlshRNA tumor cell-bearing mice with 10⁶ CD8⁺ T eell or CD8⁻ T cells intravenously via the lateral right tail vein. Tumor volume was monitored non-invasively using the Maestro™ in vivo annual Imaging system (CRI) and an electronic caliper.

in Vitro Cytotoxicity Assay

In vitro cytotoxicity was measured by the CytoTox 96 Non-Radioactive Cytotoxicity Assay according to the manufactures instructions (Promega, Madison, Wisc.), Target cells, including 4T1-controlshRNA e-GFP(+) (1.5×10⁴) cells or 4T1-controlshRNA e-GFP(−) (1.5×10⁴) cells or BNL e-GFP(−) (1.5×10⁴) cells were seeded as quintuplicate in 96-well tissue culture plates. Effector cells, CD8⁺ T cells or CD8⁻ T cells, were added to the targets at various effector/target ratios (10:1, 20:1 and 40:1) for 16 h at 37° C. Culture medium (500 μl) was recovered and incubated for 30 mln in the dark with a buffer containing NAD⁺, lactate, and tetrazolium. LDH converts lactate to pyruvate, generating NADH which reduces tetrazolium (yellow) to formazan (red), which is detected by fluorescence (490 nm). LDH release, a marker for cell death, was expressed as a percentage of the LDH in the medium over the total LDH (iysate).

Proteasome Activity Assay

Ten-million cells were lyzed using 0.5 ml cell lysis buffer (50 mM HEPES, pH7.5 5 mM EDTA, 150 mM NaCl, 1% Triton X-100 and 2 mM ATP) and incubated for 30 min on ice. Clear supernatant was recovered after centrifugation at 14,000 g for 30 min, and proteasome activity was measured using a 20S proteasome activity assay kit (Millipore Corporation) according to the manufacturers instructions. Supernatant containing 30 μg protein was incubated for 90 min at 37° C. with fluorogenic proteasome substrate, Suc-LLVY-AMC in 100 μl of the assay buffer with or without 25 μM laetacystin proteasome inhibitor. The hydroiyssed AMC was quantified using 380/460 nm filter set in a Flooroskan Ascent Flnorometer (ThermoFisher Scientific).

Statistical Analysis

For comparisons between groups, Dunn multiple comparison tests and Student's t-test and one-way analysis of variance (ANOVA) were used in this study (p values <0.001 were considered significant).

Western Blot Analysis

Total cell extracts (50 μg) from 4T1-controlshRNA and 4T1-Hsp25shRNA cells were isolated according to standard protocol (Cell Signaling, Dauvers, Mass.) and fractionated by electrophoresis on 10% SDS-PAGE and electrohfofied to PVDF membrane (GE Healthcare, Pittsburgh, Pa.) and probed with anti-Hsp25 (Santa Cruz Biotechnologies, Santa Cruz, Calif.), anti PA28α and anti-prohibitin (Cell Signaling). Protein loading control was used as β-actin-(Abcam, San Francisco, Calif.). Appropriate secondary antibodies were purchased from (Santa Cruz) were used in the study.

RNA Isolation and Real-Time PCR Analysis

Total RNA was isolated from 4T1-contolshRNA and 4T1Hsp25 shRNA cells using Qiagen RNeasy kit (Qlagen, Valencia, Calif.). Oligo-dT primed 5 μg of total RNA was converted into cDNA according to manufacturer's protocol (SA Biosciences, Frederick, Md.). Real-time PCR was performed using gene specific primers purchased from SA Biosciences.

Two-Dimensional SDS-PAGE

4T1-controlshRNA and 4T1-Hsp25shRNA cells were lyssed using lysis buffer (containing 8 M urea, 4% CHAPS, 50 mM DTT and 0.5% IPG buffer: GE Healthcare), supplemented with protease inhibitors (Roche, Indianapolis, Ind.) and halt-phosphatase Inhibitors (ThermoFisher Scientific, Rockford, Ill.). Isoelectric focusing was carried out using pH 3-10 NL, pH 4-7 NL, 11 cm. IPG strips (GE Healthcare) for 30,000 Vhrs at room temperature using the IPG 3 Ettan unit (GE Healthcare). The focused IPG strips were equilibrated in a second dimension sample buffer (25 mM Tris (pH6.8) containing 20% glycerol, 2% SDS, 2% DTT) for 15 mm, and equilibrated with the same buffer containing 2.5% of iodoaeetamide (IAA) for a further 15 min. The second dimension gel electrophoresis was performed on 8-16% polyacrylamlde gradient SDS gel (Bio-Rad, Hercules, Calif.) and the samples were electropboresed until the dye front reached the opposite end of the gel. The gel was then fixed for 20 h with fixing solution containing 50% ethanol and 1% phosphoric acid. Thereafter, gels were stained with Bio-Safe Coomassic Blue Stain (Bio-Rad) and destained with high-grade deionized water (Milllpore Corporation, Billerica, Mass.) water to remove the background staining.

Mass/Spectromeitric Analysis of Tryptic Peptides

The gel spots were cut using Bio-Rad's EXQuest Spot Cutter and proteins were digested in-gel, and peptides were extracted, and analyzed, as described earlier (Bhai 2005).

Flow Cytometry

Flow cytometry was used for the analysis and sorting of GFP signals using a BD FACSAria flow cytometer (BD Biosciences, San Jose, Calif.) equipped with a 488 nm argon laser. The emission filter for GFP was set to 515-545 nm. For GFP sorting, 4T1-controlshRNA and 4T1-Hsp25shRNA cells were harvested and suspended in PBS buffer containing 2% PBS to a concentration of 10⁷ cells/ml. Cells were appropriately gated by forward/size scatter and 2-3% cells gated events were collected per sample. Post sorted cells were collected in cell culture medium containing 20% FBS and plated in 4T1 complete media.

Haematoxylin & Eosin (H&E) Analysis, Immunohistochemical Staining and Fluorescence Microscopy

At the end of the experiment, animals were sacrificed using euthasol injection. The lungs, heart, liver, kidneys, brain, spleen and hind limbs were incised and fixed in 10% formalin. All tissues were embedded in paraffin. Histological sections were prepared by standard conventional processing and stained with H&E and digital pictographs were taken using an Olympus CKX41 microscope equipped with a DP71 CCD camera (Olympus, Center Valley, Pa.). Standard fluorescence microscopy was performed using the same microscope. Phase contrast and GFP fluorescence images were captured with DP71 image acquisition interface software (Olympus).

Clonogcolc Assay

Lung metastasis was determined using the clonogenic assay as previously described (Bansero, 2004), Lung tissue (n=5) was aseptically removed, minced with trypsin and seeded in triplicate after dilution series (1:20 to 1:320) in 60-mm3 Petri dishes and incubated for 10-12 days at 37° C. Plates were then washed twice with PBS and colonies were visualised and counted alter staining with crystal violet.

### Example 2

### Hsp25shRNA Inhibits Tumors, Results

Hsp25shRNA Permanently Silences hsp25 Gene Expression

A lentivirus-based vector (pLVTHM) was used that expresses RNAi inducing the twenty-five kilo Dalion heat shock protein (Hsp25)shRNA (Hsp25shRNA) under the control of the H1 promoter (FIG. 1A). This bicistronic vector was engineered to coexpress enhanced green fluorescent protein (GFP) as a reporter gene under the tight control of the elongation factor-1 alpha (EF-1α) promoter, permitting transduced/infected target cells to be tracked using in vivo imaging. Stable silencing of hsp25 gene expression in 4T1 tumor cells was achieved by subcloning the Hsp25shRNA cassette into pLVTHM, a self-inactivating (SIN) ientiviral vector using Mlu I and Cla I restriction sites (4T1-Hsp25shRNA hairpin loop sequence) (FIG. 1A). A control/scrambled shRNA was also constructed containing Ientiviral vector which does not have sequence homology to the mouse genome (4T1-controlshRNA hairpin loop sequence) (FIG. 1A). These constructs were introduced into 293FT viral packaging cells to make lentivirus. The concentrated lentivirus preparation was used to infect target 4T1 breast adenocarcinoma cells. The resulting GFP expression was assessed 4 days post infection by flow cytometry and further enriched for only highly expressing GFP-posihve cells. The resulting sorted 4T1-Hsp2SshRNA cells were 96.7% positive for GFP (FIG. 1B). The high GFP expression exhibited by both 4T1-controlshRNA and Hsp25shRNA stable transacted cells remained high even after 6 weeks of culture (FIG. 1C). High GFP expression was confirmed in 4T1-Hsp25shRNA cells corresponded to efficient silencing of Hsp25 protein expression consistently by >98% after 6-8 weeks in vitro cell culture (FIG. 1D).

Silencing Hsp25 Protein Increases Tumor Cells Death and Increases the Ability of Tumors to Migrate in vitro

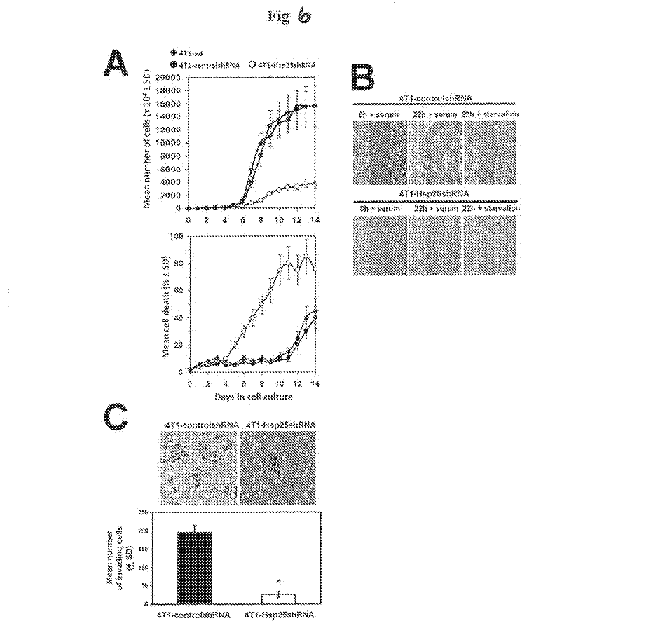

The uncontrollable growth of tumors and their ability to metastasize and invade distant organs is a serious problem. Silencing Hsp25 protein expression drastically suppressed the proliferative capacity of 4T1 Hsp25shRNA cells (FIG. 6A; top panel open circles) as compared to control cells (4T1-controlshRNA) (FIG. 6A; top panel filled circles) or wild type 4T1 (4T1-wt) cells (FIG. 6A; top panel filled diamonds). Results of cell death measurements (FIG. 6A; bottom, panel) suggests that loss of proliferative capacity is due to a concomitant increase in cell death (FIG. 6A; bottom panel open circles), as compared, to 4T1-controlshRNA (FIG. 6A; bottom panel filled circles) or 4T1-wt (FIG. 6A; bottom panel filled diamonds). We demonstrated that Hsp25shRNA treatment adversely affects the directional cell migration of 4T1 cells in vitro, almost to the same extent as serum starvation, as judged by the wound healing experiment (FIG. 6B). These results correlated well with the inability of 4T1-Hsp25shRNA cells to invade extracellular matrix in vitro as compared to 4T1-controlshRNA cells (FIG. 6C). Silencing the hsp25 gene significantly downregulated the expression of MMP-9 as compared to 4T1-controlshRNA cells (data not shown). The expression of additional genes involved in cell survival, migration and metastasis, including COX2, PAR1, TWIST ID1 and SPARC were amplified by RT-PCR; however, no significant differences in gene expression levels were observed between 4T1-controlshRNA and 4T1-Hsp25shRNA cells. Together, these results indicate that silencing the expression of Hsp25 in 4T1 breast adenocarcinoma tumors interferes with its ability to proliferate and metastasize in vitro.

High Expression of Hsp25 Represses Proteasome Activity and Tumor Suppressor Genes

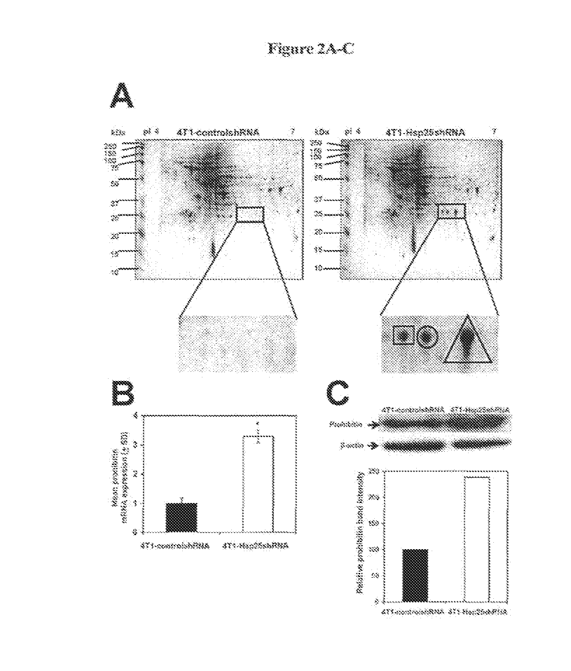

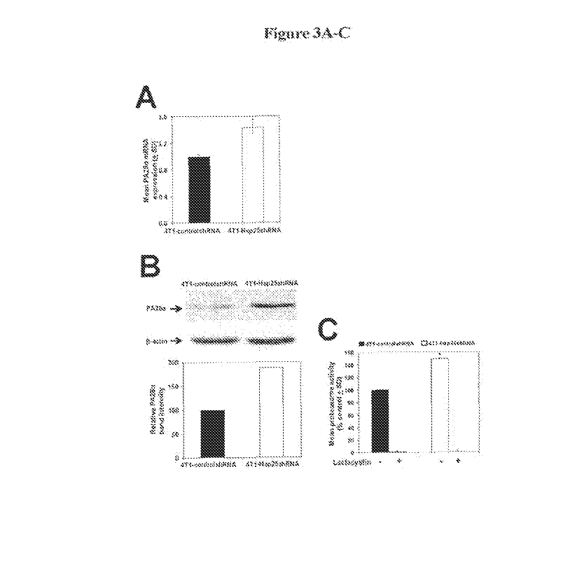

To obtain an integrative understanding on the effect of Hsp25 silencing on protein expression in 4T1 breast adenocarcinoma cells, 2D SDS-PAGE was combined with LC-MS/MS techniques to compare the protein profiles between controlshRNA and Hsp25shRNA stably transfected 4T1 cells. Three unique spots were selected from 4T1-Hsp25shRNA cells (FIG. 2A; right panel) which were absent in 4T1-controlshRNA cells (FIG. 2A; left panel). Further characterization using LC-MS/MS and bioinformatics revealed that the unique proteins were NG,Ng-dimethylarginine dimethylaminohydrolase 2 and prohibitin (Table I; square), PA28α, PA28γ and mitochondrial ribosomal protein L46 (Table I; circle). Proteins expressed within the triangle could not be identified, possibly due to the highly glycostasis nature of the proteins (Table I; triangle). Due to the obvious relevance to tumor growth and metastasis, we chose to validate prohibitin and PA28α by real-time PCR and Western blot analysis. We demonstrated that silencing the hsp25 gene increased prohibitin mRNA expression by 3-fold (FIG. 2B). mRNA expression levels correlated well with a 2.5-fold increase in prohibitin protein expression as judged by Western blot analysis (FIG. 2C). Similar increases were observed for PA28α mRNA expression which was upregulated by 1.5-fold, as judged by real-time PCR (FIG. 3A) and by 2-fold as judged by Western blot analysis, as compared to controls (FIG. 3B). There was no significant alteration in PA28γ protein and RNA levels. To further validate the findings that silencing Hsp25 protein expression increases the proteasome activity, we measured the chymotrypsin-like activity of 20 S proteasome in 4T1-controlshRNA and 4T1-Hsp25shRNA cell extracts. We demonstrated that 4T1-Hsp25shRNA cells showed 50% more proteasome activity than 4T1-controlshRNA tumor cells (FIG. 3C). Together, these results indicate that silencing of Hsp25 enhances the tumor suppressor gene prohibitin and proteasome function via PA28α.

Silencing Hsp25 Expression Induces Tumor Regression and Inhibits Metastasis

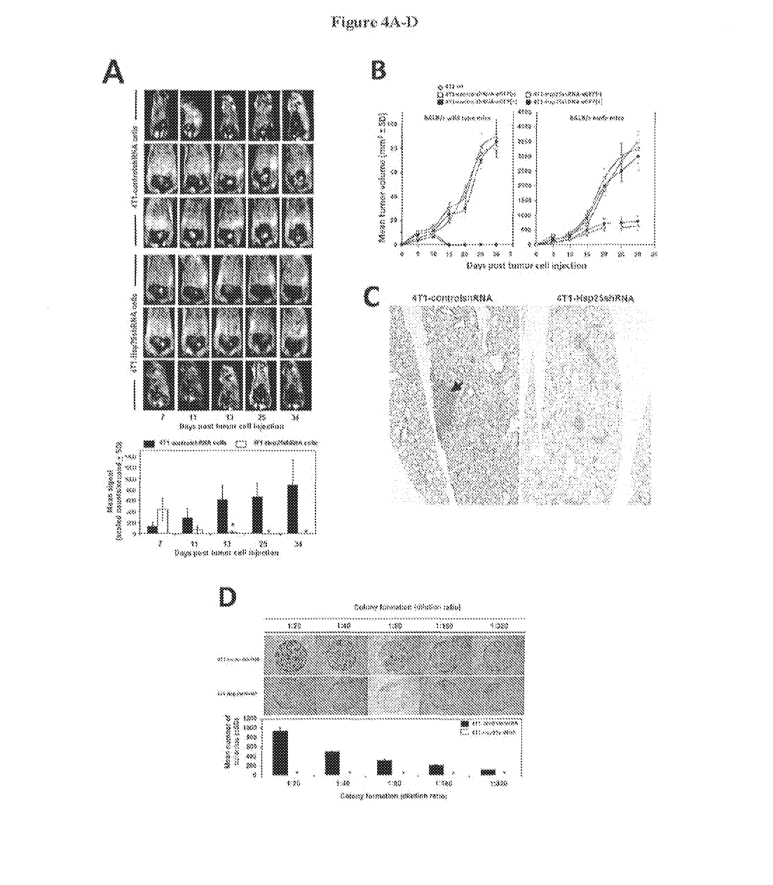

To determine the consequence of lentivirus-mediated hsp25 gene silencing in vivo, 4T1-controlshRNA and 4T1-Hsp25shRNA tumor cells were injected subcutaneously (s.c) into the mammary pad of female BALB/c mice. As early as 7 days post tumor cells injection (TCI), tumors could be visualized growing in the mammary pad of all mice. Mice injected with 4T1-controlshRNA tumors grew progressively and were sacrificed by day 34 past TCI, due to the tumor burden (FIG. 4A). In contrast, mice injected with 4T1-Hsp25shRNA tumor cells demonstrated a steady regression of tumors alter day 7 post tumor cell inoculation with no detectable GFP signal after day 25 (FIG. 4A). Efficient Hsp25 silencing (>95%) could still be demonstrated in 4T1-Hsp25shRNA tumor before they completely disappeared (day 13 post tumor cell injection). To determine whether the anti-tumor responses was directed against the GFP protein instead of unknown “tumor-associated” antigen that are better processed as a consequence of Hsp25 down modulation in tumor cells, tumor growth experiments were performed using eGFP positive(+) and negative(−) 4T1-Hsp25shRNA and 4T1-controlshRNA, and wild type 4T1 cells. We demonstrate that eGFP did not significantly alter tumor growth curves (FIG. 4B). Experiments performed in BALB/c nude mice reveal that the growth kinetics of 4T1-Hsp25shRNA cells is indeed slower than 4T1-controlshRNA or 4T1 wild type cells (FIG. 4B; right panel). An additional observation in nude mice was that whereas 4T1-controlshRNA and 4T1 wild type cells rapidly metastasize to distant organs including lungs, liver and brain, 4T1-Hsp25shRNA cells do not metastasize to these organs suggesting that a competent immune system (possibly CD8⁺ CTL) is required to control metastasis.

At the end of the experiment (day 34 post TCI), gross pathology of multiple organs, including lungs, brain, bone and liver demonstrated an absence of tumor metastasis in mice injected with 4T1-Hsp25shRNA, but not 4T1-controlshRNA mice, H&E staining of lungs from mice injected with 4T1-controlshRNA revealed micrometastasis in lung tissues (FIG. 4C; left panel). In contrast, lungs of mice injected with 4T1-Hsp25shRNA had no visible micrometastasis (FIG. 4C; right panel). To confirm that micrometastasis undetectable by light microscopy did not exist in 4T1-Hsp25shRNA injected mice; we performed colonogenisity assays on lung tissues in the presence of complete media containing 6-thioguanine. 4T1 breast adenocarcinoma cells are resistant to 6-thiognanine, however, all other contaminating cells will be destroyed, Mice injected with the 4T1-controlshRNA cells exhibited large numbers of colonies at all: dilution, reflecting robust metastasis of tumors to the lungs (FIG. 4D). In contrast, no colonies were observed in dishes plated with lung tissue harvested from mice injected with 4T1-Hsp25shRNA cells (FIG. 4D). Together, these data suggest that permanent silencing of Hsp25 results in tumor regression and inhibition of metastasis in vivo.

Silencing Hsp25 Activates Specific CD8⁺ Cytotoxic T Lymphocyte (CTL) Killing Functions

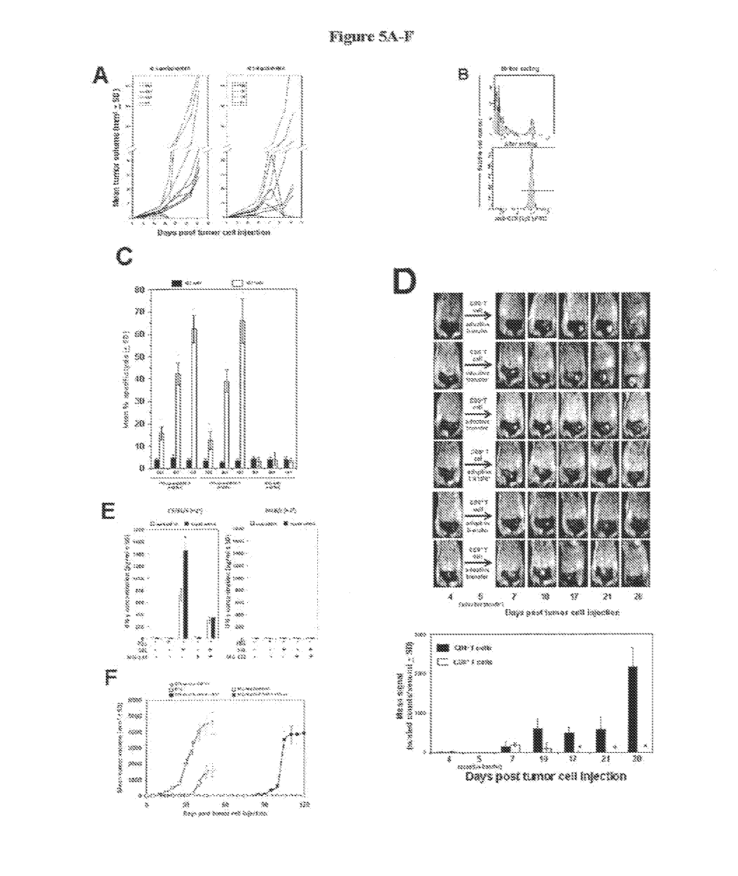

To determine the nature of the cells responsible for tumor regression following silencing of Hsp25 expression in 4T1 breast adenocarcinoma cells, prior to TCI, we performed in vivo depletion of cells known to play an important role in tumor regression. Here, we demonstrated that in vivo depletion of CD8⁺ CTL prior to injection with 4T1-controlshRNA cells (FIG. 5A; left panel, red lines), drastically increased tumor growth rate and by day 34 post TCI the size of the tumors were approximately 10 times larger than mice injected with PBS only (FIG. 5A; left panel, black lines). The in viva depletion of CD4⁺ T cells did not significantly alter tumor growth rate or tumor volume in mice Injected with 4T1-controlshRNA cells (FIG. 5A; left panel, blue lines). Unexpectedly, using similar mice the in vivo depletion of NK cells using the 5E6 monoclonal antibody induced complete tumor regression (FIG. 5A; left panel, green lines). In mice injected with 4T1-Hsp25shRNA cells, no tumor growth was seen in any of the mice by the end of the experiment (FIG. 5A; right panel, black lines). As expected, the in vivo depletion of CD8⁺ T cells (FIG. 5A: right panel, red lines) and NK cells (FIG. 5A; right panel, green lines), prior to injection with 4T1-Hsp25shRNA cells resulted in tumor growth. Similar depletion of CD4⁺ T cells initially resulted in increased tumor growth, followed, by tumor regression (FIG. 5A; right panel, blue lines). Interestingly, although the in vivo depletion of CD8⁺ T cells prior to injection with 4T1-Hsp25shRNA cells resulted in increased tumor growth (FIG. 5A; right panel, red lines), gross pathology of lung, brain and bone did not reveal any signs of metastasis to the lungs. Similarly, injection of 4T1-Hsp25shRNA cells into the breast pad of BALB/c nude mice resulted in tumor growth without metastasis.

To confirm that CD8⁺ T cells mediated the enhanced cytolytic effects after silencing Hsp25, reactive CD8⁺ T cells were harvested from the spleen of mice which had been injected with 4T1-Hsp25shRNA cells and were tumor-free (days 21-28 post TCI) and the specific T-cell cytotoxicity measured against 4T1-controlRNA target cells ex vivo. Extracted splenic CD8⁺ T cells were enriched using negative selection by magnetic beads and consistently exhibited >95% purity, as judged by flow cytometry (FIG. 5B). Experiments were next performed to negate the possibility that the tumor associated response was directed against GFP protein. We demonstrated that reactive CD8⁺ T cells, but not CD8⁺ T cells (non-CD8⁺ T cells) effector cells harvested from the spleen of mice injected wtih 4T1-Hsp25shRNA cells exhibited potent-specific lysis against 4T1-controlshRNA e-GFP positive and e-GFP negative targets with similar activity (FIG. 5C). CD8⁺ cells did not exhibit significant lytic activity against BNL, which served as an irrelevant target (FIG. 5C). As expected, both CD8⁺ and CD8⁻ T cells from mice injected with 4T1-controlshRNA cells did not mediate significant lysis above base-line levels against 4T1-controlshRNA targets.

To determine whether 4T1-Hsp25shRNA reactive CD8⁺ T cells could rescue mice injected with 4T1-controlshRNA cells, 4T1-Hsp25shRNA reactive CD8⁺ T cells were adoptively transferred into 4T1-controlshRNA tumor-bearing mice. As predicted, the adoptive transfer of 4T1-Hsp25shRNA reactive CD8⁺ T cells into 4T1-controlshRNA tumor-bearing mice induced significant tumor regression starting by day 17 post TCI and by day 28 there was no detectable tumor growth (FIG. 5D). In contrast, 4T1-controlshRNA tumor-bearing mice adoptively transferred with CD8⁺ T cell fraction were not protected and mice rapidly developed tumors (FIG. 5D) and metastasis.

To demonstrate that the improvement in antigen presentation is due to silencing Hsp25expression, we used the in vitro cross-presentation assay. BMDC were recovered from female C57BL/6 (B2^{b}) and BALB/c (H2^{d}) mice and treated with OVA during the culture process. BMDC were then transfected with either Hsp25-siRNA or negative control-siRNA and fixed with paraformaldehyde, and later admixed with S8L peptide-specific T cell hybridoma, B3Z. We demonstrate that B3Z cells released significantly more IFN-γ when admixed with C57BL/6 (H2^{b})-derived BMDC in which Hsp25 has been silenced (Hsp25-siRNA), as compared to control-siRNA treated BMDC (FIG. 5E; left panel). In addition, we demonstrate that pre-treatment of both Hsp25-siRNA- and control-siRNA-treated BMDC with the specific proteasome inhibitor MG-132, significantly reduced the concentration of released IFN-γ (FIG. 5E; left panel). Finally, we demonstrate that BMDC recovered from BALB/c mice which express H2^{d} did not release significant quantities of IFN-γ under similar conditions (FIG. 5E: right panel). To prove that 4T1-Hsp25shRNA generates memory responses, tumor-free immunocompetent female BALB/c mice were re-challenged with wild type 4T1 (4T1-wt) or an irrelevant tumor, murine transformed hepatocellular carcinoma (HCC) cells, BNL, 60 days post initial challenge with 4T1-Hsp25shRNA. We demonstrate that re-challenge of 4T1-wt cells does not result in tumor growth (FIG. 5F; filled circles), which is similar to mice injected with 4T1-Hsp25shRNA alone (FIG. 5F; open circles). However, re-challenge with BNL (after 4T1-Hsp25shRNA) resulted in tumor growth (FIG. 5F; filled squares) in a similar fashion to mice injected with BNL alone (FIG. 5F; open squares).

TABLE I
Identification of unique proteins in lentivirus-mediated
Hsp25 knockdown of 4T1 cells by mass spectrometry.

| | | Database | Distinct summed | | % Amino | |
|---|---|---|---|---|---|---|
| 2D-Gel | | accession | MS/MS search | Protein MW | acid | Number of |
| spot^{a} | Protein name | number | score | (kDa)/pI | coverage | peptides |
| Square | NG, Ng- | 45476968 | 81.94 | 29,646/5.66 | 27 | 5 |
| | dimethylarginine | 74181431 | 65.46 | 29,850/5.40 | 21 | 5 |
| | dimethylaminohydrolase | | | | | |
| | 2 | | | | | |
| Circle | Prohibitin | 12842740 | 168.86 | 28,640/5.48 | 50 | 41 |
| | Proteasome (prosome, | 6755214 | 80.66 | 29,506/5.69 | 34 | 6 |
| | macropain) 28 subunit | | | | | |
| | alpha, PA28α | | | | | |
| | Proteasome activator | 12963643 | 62.77 | 32,131/6.93 | 16 | 5 |
| | subunit 3 | | | | | |
| Triangle | Mitochondrial | — | — | — | — | — |
| | ribosomal | | | | | |
| | protein L46 | | | | | |
| | Not detectable | | | | | |

^{a}4T1-controlshRNA or 4T1-Hsp25shRNA cells were run on 2D-SDS PAGE and protein spot was excised using Bio-Rad's ExQuest spot cutter. Protein sample was digested in-gel, and peptides extracted and samples injected into a 1100 series HPLC-Chip cube MS interface, and Agilent 6300 series Ion Trap Chip-LC-MS/MS system (Agilent Technologies). The system is equipped with a HPLC-Chip (Agilent Technologies) that incorporates a 40 nL enrichment column and a 43 mm × 75 mm analytical column packed with Zorbex 300SB-C18 5 mm particles. Tandem MS spectra were searched against the National Center for Biological information nonredundant (NCBInr) mouse protein database, using Spectrum Mill Proteomics Work Bench for protein identification.

### Example 3

### CH101 in Combination with Chemotherapeutic Drugs

CH101 is a new generation of anticancer drugs based on interference RNA (RNAi) technology. CH101 is a cocktail of two dsRNA molecules, dsRNA SEQ ID NO:8/SEQ ID NO:9 and dsRNA SEQ ID NO:10/SEQ ID NO:11, CH101 functions by blocking the action of heat shock protein-27 (Hsp27), known to be highly expressed in certain cancers and demonstrated to confer resistance to chemoiherapeutic agents through its anti-apoptotic actions. CH101 concomitantly increases tumor's proteasome function, which in turn results in efficient antigen presentation and stimulates cytotoxic T lymphocyte (CD8⁺ T cell) memory and tumor killing functions.

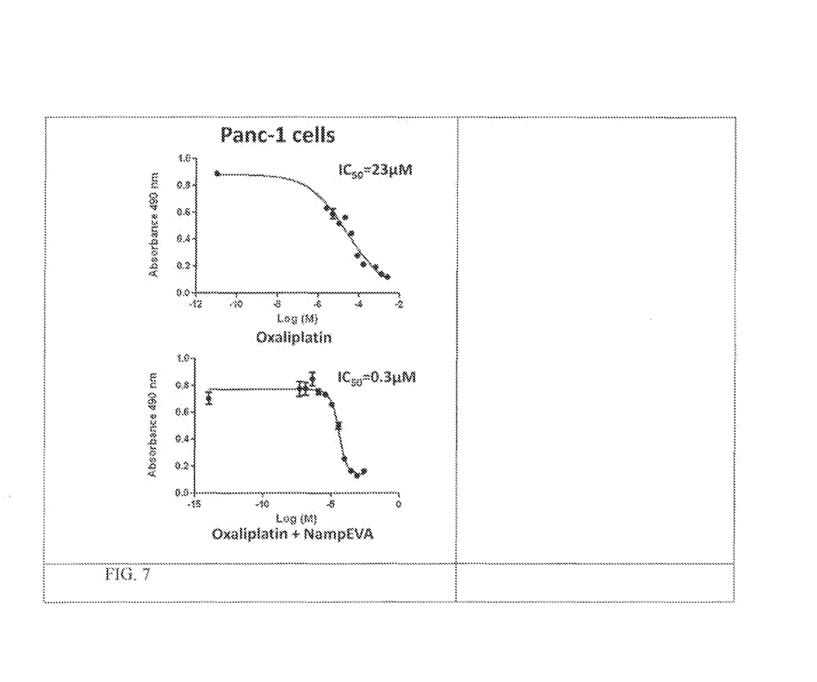

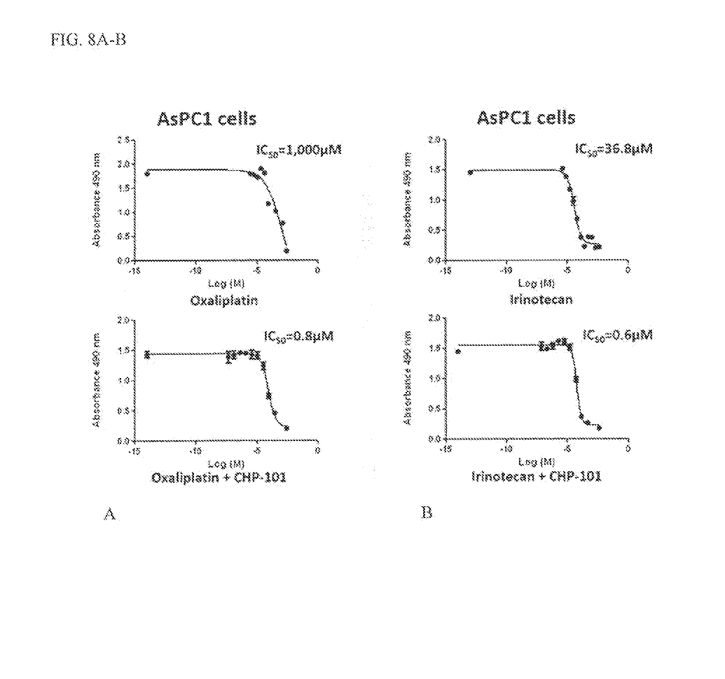

It has been demonstrated in this study that CH101 is more effective against highly metastatic cancers (MDA-MB-231; breast cancer and AsPC1; pancreatic cancer) than non-metastatic or weakly metastatic cancers (MCF7; breast cancer and Panc-1; pancreatic cancer) (FIG. 8A). In addition, CH101 in combination with certain chemotherapeutic drugs functions synergistically to kill tumors. It has been demonstrated thai the IC₅₀ for the chemotherapeutic drug oxaliplatin for the weakly metastatic pancreatic cancer cell is 23 μM (FIG. 7; top panel). Combined oxaliplatin +CH101 treatment reduced the IC₅₀ by 100-fold to 0.3 μM (FIG. 7; bottom panel).

It has been further demonstrated that in the highly aggressive, highly metastatic pancreatic cell, AsPC1, the IC₅₀ for the chemotherapeutic drug oxaliplatin is 1,000 M (FIG. 8A; top panel). The combined treatment with oxaliplatin +CH101 reduced the IC₅₀ by 10,000-fold to 0.8 μM (FIG. 8A: bottom panel). Also, the combined treatment of CH101 and irinotecan in AsPC1 showed an IC₅₀ of 0.6 μM (FIG. 8B, bottom panel). Adding CH101 to the treatment regime of platinum chemotherapy agents will results in superior anti-cancer treatment and may drastically reduce the dose of chemotherapy required to eradicate cancer and by extension the chemotherapy associated side effects.

Oxaliplatin is an analog of cisplatin, the first successful platinum-containing anticancer drug. It is one of the so-called DACH (1,2-Diamincyclohexane)-containing platinum complexes that exhibited activity in Murine L1210 leukemia tumor models possessing acquired resistance to cisplatin. These platinum-containing drugs interfere with the genetic material, or DNA, inside the cancer cells and prevent them from further dividing and growing more cancer cells. Oxaliplatin has been used to treat metastatic colorectal cancer, and advanced ovarian cancer and has been tested with some results in head and neck cancers, skin cancer, lung cancer, and non-Hodgkins lymphomas. Platinum chemotherapeutic agents have been the treatment of choice for ovarian cancer for the past twenty years. Now they are also proving effective against certain other cancers including testicular, bladder, endometrial, colon, and lung cancer and some cancers of the head and neck. Side effects include peripheral neuropathy, nausea and vomiting, diarrhea, fatigue, loss of appetite, mouth sores, low blood counts which increases risk for Infection, anemia and/or bleeding.

In a parallel experiment, it has been demonstrated that combined CH101 was effective in reducing the IC₅₀ of irinotecan from 36.8 μM to 0.6 μM in the highly aggressive, highly metastatic pancreatic cell, AsPC1. However, combined CH101 plus irinotecan was not effective in reducing the IC₅₀ of the weakly metastatic pancreatic cell, Panc-1.

Irinotecan (Camptosar, Pfizer; Campto, Yakult Honsha) is a drag used for the treatment of cancer. Irinotecan is a topoisomerase 1 inhibitor, which prevents DNA from unwinding. In chemical terms, it is a semisynthetic analogue of the natural alkaloid camptothecin. The most significant adverse effects of irinotecan are severe diarrhea and extreme suppression of the immune system. The immune system is adversely impacted by irinotecan. This is reflected in dramatically lowered white blood cell counts in the blood, in particular the neutrophils. The patient may experience a period of neutropenia (a clinically significant decrease of neutrophils in the blood) while the bone marrow increases white cell production to compensate.

Taken together, these data demonstrate that CH101 is more effective against highly metastatic cancers (MDA-MB-231; breast cancer and AsPC1; pancreatic cancer) than non-metastatic or weakly metastatic cancers (MCF7; breast cancer and Panc-1; pancreatic cancer). In addition, that combination of CH101 with platinum chemotherapy agents will results in superior anti-cancer treatment and will drastically reduce the dose of chemotherapy required to eradicate cancer and by extension the chemotherapy associated side effects. However, CH101 in combination with topoisomerase 1 inhibitors should only be used for more advanced highly metastatic disease.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein, while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Bhat V B, Choi M H, Wishnok J S, Tannenbaum S R. Comparative plasma, proteome analysis of lymphoma-bearing SJL mice, J Proteome Res 2005;4:1814-1825.
Bausero M A, Page D T, Osinaga E, Asea A, Surface expression of Hsp25 and Hsp72 differentially regulates tumor growth and metastasis. Tumour Biol 2004;25:243-251.
1. Oesterreich S, Pliekey E, Weber L A, Fuqua S A, Basal regulatory promoter elements of the hsp27 gene in human breast cancer cells. Blochem Biophys Res Commun 1996;222:155-163.
2. Egeblad M, Werb Z. New functions for the matrix metalloproteinases in cancer progression, Nat Rev Cancer 2002;2:161-174.
3. Soldes O S, Knick R D, Thompson I A, 2nd, Hughes S J, Orringer M B, Iannettoni M D, Hanash S M, Beer DG. Differential expression of Hsp27 in normal oesophagus, Barrett's metaplasia and oesophageal adenocarcinomas. Br I Cancer 1999;79:595-603.
4. Budhram-Mahadeo V S, Heads R J Heat shock protein-27 (hsp27) in breast cancers: regulation of expression and function. In: S K Calderwood, M Y Sherman, D R Ciocca editors. Heat Shock Proteins In Cancer. Dordrecht, The Netherlands; Springer; 2007. 93-130
5. O'Neill P A, Shaaban A M, West C R, Dodson A, iarvis C, Moore P, Davies M P, Sibson D R, Foster C S. Increased risk of malignant progression in benign proliferating breast lesions defined by expression of heat shock protein 27. Br J Cancer 2004;90:1:82-188.
6. Rust W, Ksngsley K, Petnieki T, Padmaoabhan S, Carper S W, Plopper G E. Heat shock protein 27 plays two distinct roles in controlling human breast cancer cell migration on Iaminin-5. Mol Cell Biol Res Commun 1999;1:196-202.
7. Ciocca D R, Lo Castro G, Alonio L V, Cobo M F, Lotfi H, Teyssie A. Effect of human papillomavirus infection on estrogen receptor and heat shock protein hsp27 phenotype in human cervix and vagina. int J Gynecol Pathol 1992;11:113-121.
8. Oesterreich S, Weng C N, Qiu M, Hilsenbeck S G, Osborne C K, Fuqua S A. The small heat shock protein hsp27 is correlated with growth and drug resistance in human breast cancer cell lines. Cancer Res 1993;53:4443-4448.
9. Yamamoto K, Okanaoio A, Isonishi S, Ochiai K, Ohtake Y. Heat shock protein 27 was up-regulated in cisplatin resistant human, ovarian tumor cell line and associated with the cisplatin resistance. Cancer Lett 2001-168:173-181.
10. Storm F K, Mahvi D M, Gilchrist K W. Pleat shock protein 27 over/expression in breast cancer lymph node metastasis. Ann Surg Oncol 1996;3:570-573.
11. Thor A, Benz C, Moore D, 2nd, Goldman E, Edgerton S, Landry J, Schwartz L, Mayall B, Hickey E, Weber L A. Stress response protein (srp-27) determination in primary human breast carcinomas; clinical, histologic, and prognostic correlations. J Natl Cancer Inst 1991;83:170-178.
12. Vargas-Roig L M, Gago F E, Tello O, Aznar J C, Ciocca D R. Heat shock protein expression and drug resistance in breast cancer patients treated with induction chemotherapy. International Journal of Cancer 1998;79:468-475.
13. Sondel P M, Rakhmilevich A L, de-Jong JLO, Hank J A Cellular immunity and cytokines, in: J Mendelsohn, PM Howley, M A Israel, L A Liotta editors. The Molecular Basis of Cancer. Philadelphia; W. B. Saunders; 2001. 535-571
14. Kloetzel P M. The proteasome and MHC class I antigen processing. Biochim Biophys Acta 2004;1695:225-233.
15. Groettrup M, Soza A, Eggers M, Kuehn L, Dick T P, Schild H, Rammensee H G, Knszinowski U H, Kloetzel P M. A role for the proteasome regulator PA28alpha in antigen presentation. Nature 1996;381:166-168.
16. Dick T P, Ruppert T, Groettrup M, Kloetzel P M, Kuehn L, Koszinowski U H, Stevanovic S, Schild H, Rammensee H G, Coordinated dual cleavages induced by the proteasome regulator PA28 lead to dominant MHC ligands. Cell 1996;86:253-262.
17. Sun Y, Sijts A J, Song M, Janek K, Nussbaum A K, Kral S, Schirle M, Stevanovic S, Paschen A, Schild H, Kloetzel P M, Schadendorf D. Expression of the proteasome activator PA28 rescues the presentation of a cytotoxic T lymphocyte epitope on melanoma cells. Cancer Res 2002;62:2875-2882.
18. Stohwasser R, Salzmann U, Giesebrecht J, Kloetzel P M, Holzhutter H G. Kinetic evidences for facilitation of peptide channelling by the proteasome activator PA28. Eur J Biochem 2000;267:6221-6230.
19. Whitby F G, Masters E I, Kramer L, Knowlton J R, Yao Y, Wang C C, Hill C P. Structural basis for the activation of 20S proteasomes by 11S regulators, Nature 2000;408:115-120.
20. Pamer E, Cresswell P. Mechanisms of MHC class I-restricted antigen processing. Annu Rev Immunol 1998;16:323-358.
21. Naldini L, Blomer U, Gallay P, Ory D, Mulligan R, Gage F H, Verma I M, Trono D. In vivo gene delivery and stable transduction of nondividing cells by a lentiviral vector. Science 1996;272:263-267.
22. Bhat V B, Choi M H, Wishnok J S, Tannenbaum S R. Comparative plasma proteome analysis of lymphoma-beadng SJL mice, J Proteome Res 2005;4:1814-1825.
23. Bausero M A, Page D T, Osinaga E, Asea A. Surface expression of Hsp25 and Hsp27 differentially regulates tenor growth and metastasis. Tumour Biol 2004;25:243-251.
24. Chauhan D, Li G, Shringarpure R, Podar K, Ohtake Y, Hideshima T, Anderson K C. Blockade of Hsp27 overcomes Bortezomib/proteasome Inhibitor PS-34I resistance in lymphoma cells. Cancer Res 2003;63:6174-61.
25. Mitsiades N, Mitsiades C S, Poulaki V, Chauhan D, Fanourakis G, Gu X, Bailey C, Joseph M, Libermann T A, Treon S P, Munshi N C, Richardson P G, Hideshima T, Anderson K C. Molecular sequelae of proteasome inhibition in human multiple myeloma cells, Proc Natl Acad Sci USA 2002;99:14374-14379.
26. Guay J, Lambert H, Gingras-Breton G, Lavoie J N, Hunt J. Landry J. Regulation of actin filament dynamics by p38 map kinase-mediated phosphorylation of heat shock protein 27. J Cell Sci 1997;110 (Pt 3):357-368.
27. Yu Y Y, George T, Dorfman J R, Roland J, Kumar V, Bennett M. The role of Ly49A and 5E6(Ly49C) molecules in hybrid resistance mediated by murine natural killer cells against normal T cell blasts. Immunity 1996;4:67-76.
28. Koh C Y, Blazar B R, George T, Welniak L A, Capitini C M, Raziuddin A, Murphy W J, Bennett M. Augmentation of antitumor effects by NK cell inhibitory receptor blockade in vitro and in vivo. Blood 2001;97:3132-3137.
29. Farooqui-Kabir S R, Budhram-Mahadeo V, Lewis H, Latchman D S, Marber M S, Heads R J. Regulation of Hsp27 expression and cell survival by the POU transcription factor Brn3a. Cell Death Differ 2004;11:1242-1244.
30. Roechi P, Beraldi E, Ettinger S, Fazli L, Vessella R L, Nelson C, Gleave M. Increased Hsp27 after androgen ablation facilitates androgen-independent progression in prostate cancer via signal transducers and activators of transcription 3-mediated suppression of apoptosis. Cancer Res 2005;65:11083-11093.
31. Rocchi P, Jugpal P, So A, Sinneman S, Ettinger S, Fazli L, Nelson C, Gleave M, Small interference RNA targeting heat-shock protein 27 inhibits the growth of prostatic cell lines and induces apoptosis via caspase-3 activation in vitro. BJU Int 2006;98:1082-1089.

References include those from U.S. PATENT APP. PUB 20100186102, which is hereby Incorporated by reference.

1. A method of treating a subject with metastatic cancer or at risk of developing metastatic cancer, said method comprising administering to said subject with metastatic cancer or at risk of developing a metastatic cancer a pharmaceutically effective amount of a composition comprising an isolated double stranded ribonucleic acid (dsRNA) molecule that inhibits the expression of heat shock protein-27 (Hsp-27) using ribonucleic acid interference (RNAi) technology.

2. The method of claim 1, wherein said subject is a human subject.

3. The method of claim 1, wherein said subject has breast cancer.

4. The method of claim 3, wherein said breast cancer is ER-positive, PgR-positive and Her2-neu-negative.

5. The method of claim 3, wherein said breast cancer is ER-negative, PgR-negative and HER2/neu-positive.

6. The method of claim 3, wherein said subject has breast cancer that has undergone metastasis.

7. The method of claim 1, wherein said subject has pancreatic cancer.

8. The method of claim 7, wherein said subject has pancreatic cancer that has undergone metastasis.

9. The method of claim 1, wherein said dsRNA has a length of 19 to 28 nucleotides.

10. The method of claim 1, wherein one strand of said dsRNA comprises SEQ ID NO:3.

11. The method of claim 1, wherein said dsRNA is comprised in a vector.

12. The method of claim 11, wherein said vector is a viral vector.

13. The method of claim 12, wherein said viral vector is a retroviral vector or a lentiviral vector.

14. A method of treating a subject with cancer, comprising administering to said subject with cancer a pharmaceutically effective amount of a composition comprising an isolated dsRNA molecule that inhibits the expression of Hsp-27 and a synergistically effective amount of a platinum-containing chemotherapeutic agent.

15. The method of claim 14, wherein said platinum-containing chemotherapeutic agent is selected from the group consisting of cisplatin, carboplatin, and oxaliplatin.

16. The method of claim 14, wherein said dsRNA and said platinum-containing chemotherapeutic agent are administered concurrently.

17. The method of claim 14, wherein said dsRNA and said platinum-containing chemotherapeutic agent are administered consecutively.

18. The method of claim 14, wherein said subject has breast cancer, prostate cancer, uterine cancer, ovarian cancer, head and neck cancer, gastric cancer, brain cancer, ocular cancer, skin cancer, lung cancer, esophageal cancer, stomach cancer, liver cancer, colon cancer, rectal cancer, cervical cancer, lymphoma, leukemia, testicular cancer, bladder cancer or pancreatic cancer.

19. The method of claim 14, wherein said subject has a primary cancer that has undergone metastasis.

20. The method of claim 19, wherein said primary cancer is breast cancer or pancreatic cancer.

21. The method of claim 14, wherein said dsRNA has a length of 19 to 28 consecutive nucleotides and wherein one strand of said dsRNA comprises SEQ ID NO: 3.

22. A method of treating a subject with cancer, said method comprising administering to said subject with cancer a pharmaceutically effective amount of a composition comprising an isolated dsRNA molecule that inhibits the expression of Hsp-27 and a synergistically effective amount of a topoisomerase 1 inhibitor.

23. The method of claim 22, wherein said subject has a primary cancer that has undergone metastasis.

24. The method of claim 22, wherein said cancer is breast cancer or pancreatic cancer.

25. The method of claim 22, wherein said toposisomerase 1 inhibitor is selected from the group consisting of irinotecan, topotecan, camptothecin, and lamellarin D.

26. The method of claim 22, wherein said dsRNA has a length of 19 to 28 consecutive nucleotides and wherein one strand of said dsRNA comprises SEQ ID NO:3.

27. A method of reducing the chemotoxicity of a chemotherapeutic agent, said method comprising administering to a subject with cancer a pharmaceutically effective amount of a composition comprising an isolated dsRNA molecule that inhibits the expression of Hsp-27 concurrently with or prior to administration of a synergistically effective amount of said chemotherapeutic agent.

28. The method of claim 27, wherein said chemotherapeutic agent is a platinum-containing chemotherapeutic agent selected from the group consisting of cisplatin, carboplatin, and oxaliplatin.

29. A composition comprising an isolated dsRNA molecule with a length of 19 to 28 consecutive nucleotides, which inhibits the expression of Hsp-27, a length of from 19 to 28 consecutive nucleotides and a synergistically effective amount of a platinum-containing chemotherapeutic agent, wherein one strand of said dsRNA comprises SEQ ID NO:3.

30. The composition of claim 29, wherein said platinum-containing chemotherapeutic agent is cisplatin, carboplatin, or oxaliplatin.

31. A composition comprising an isolated dsRNA molecule with a length of 19 to 28 consecutive nucleotides, which inhibits the expression of Hsp-27, and a synergistically effective amount of a topoisomerase 1 inhibitor, wherein one strand of said dsRNA comprises SEQ ID NO: 3.

32. The composition of claim 31, wherein said topoisomerase 1 inhibitor is irinotecan, topotecan, camptothecin, or lamellarin D.

33. A method of treating a patient with cancer or at risk of developing cancer without the use of a preliminary test, comprising administering to said patient with cancer or at risk of developing cancer a pharmaceutically effective amount of a composition comprising stem cells capable of differentiating into CD8+ T lymphocytes and a pharmaceutically effective amount of a composition comprising an isolated double stranded ribonucleic acid (dsRNA) molecule that inhibits the expression of HSP-27, wherein said dsRNA is administered to said stem cells capable of differentiating into CD8+ T lymphocytes thereby increasing the ability of said stem cells capable of differentiating into CD8+ T lymphocytes to recognize tumor-specific antigens and kill cancer cells, and wherein said patient is treated.

34. The method of claim 33, wherein one strand of said dsRNA is selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, and SEQ ID NO:11.

35. The method of claim 33, wherein said stem cells are multipotent hematopoietic stem cells.

36. The method of claim 33, wherein said stem cells are autologous stem cells.

37. The method of claim 33, wherein said stem cells are allogeneic stem cells.

38. The method of claim 33, wherein said stem cells are derived from bone marrow, peripheral blood, or umbilical cord blood.

39. The method of claim 33, wherein said pharmaceutically effective amount of said composition comprising stem cells is administered prior to administration of said composition comprising dsRNA.

40. The method of claim 33, wherein said pharmaceutically effective amount of said composition comprising stem cells is administered following administration of said composition comprising dsRNA.

41. The method of claim 33, wherein said stem cells and said dsRNA are formulated in a single pharmaceutically effective composition.

42. The method of claim 33, wherein said cancer is breast cancer, prostate cancer, uterine cancer, ovarian cancer, head and neck cancer, gastric cancer, brain cancer, ocular cancer, skin cancer, lung cancer, esophageal cancer, pancreatic cancer, stomach cancer, liver cancer, colon cancer, rectal cancer, cervical cancer, lymphoma, leukemia, testicular cancer or bladder cancer.

43. A method of treating a patient with cancer or at risk of developing cancer without the use of a preliminary test, comprising administering to a patient with cancer or at risk of developing cancer a pharmaceutically effective amount of a composition comprising autologous CD8+ T lymphocytes, wherein said lymphocytes have been contacted with isolated double stranded ribonucleic acid (dsRNA) molecules that inhibits the expression of HSP-27.

44. The method of claim 43, wherein said patient has cancer, further comprising (a) administering to said patient a pharmaceutically effective amount of a composition comprising an isolated double stranded ribonucleic acid (dsRNA) molecule that inhibits the expression of HSP-27; (b) harvesting autologous CD8+ cells from said patient following (a); (c) administering a chemotherapeutic agent to said patient following (b); and (d) administering said harvested autologous CD8+ cells to said patient following (c).

45. The method of claim 43, wherein one strand of said dsRNA is selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, and SEQ ID NO: 11.

46. The method of claim 43, wherein said cancer is breast cancer, prostate cancer, uterine cancer, ovarian cancer, head and neck cancer, gastric cancer, brain cancer, ocular cancer, skin cancer, lung cancer, esophageal cancer, pancreatic cancer, stomach cancer, liver cancer, colon cancer, rectal cancer, cervical cancer, lymphoma, leukemia, testicular cancer or bladder cancer.

47. The method of claim 43, wherein said patient has a chemoresistant cancer or a cancer that has undergone metastasis.

48. The method of claim 44, wherein said cancer is breast cancer, and wherein said harvesting of said autologus CD8+ cells is performed by harvesting lymph nodes from said patient.

49. A method of inducing an immune response in a patient with a chemoresistant cancer without the use of a preliminary test, said method comprising administering to a patient with a chemoresistant cancer a pharmaceutically effective amount of CD8+ cells or stem cells capable of differentiating into CD8+ cells, wherein said CD8+ cells or stem cells have been contacted with a composition comprising an isolated double stranded ribonucleic acid (dsRNA) molecule that inhibits the expression of HSP-27.

50. A method of preventing the onset of cancer in a patient at risk for development of cancer without the use of a preliminary test, said method comprising administering to said patient a pharmaceutically effective amount of CD8+ cells or stem cells capable of differentiating into CD8+ cells, wherein said CD8+ cells or stem cells have been contacted with a composition comprising an isolated double stranded ribonucleic acid (dsRNA) molecule that inhibits the expression of HSP-27.

51. The method of claim 50, wherein said patient is administered autologous CD8+ cells.

52. The method of claim 50, wherein said patient is administered hematopoietic stem cells capable of differentiating into CD8+ cells.

53. The method of claim 50, wherein said patient has a mutation in BRCAI or BRCA2.

54. A pharmaceutical composition for inducing an immune response in a patient with cancer without the use of a preliminary test, said composition comprising stem cells capable of differentiating into CD8+ T lymphocytes and an isolated double stranded ribonucleic acid (dsRNA) molecule that inhibits the expression of HSP-27.

55. The pharmaceutical composition of claim 54, wherein one strand of said dsRNA is selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, and SEQ ID NO:11.

56. A pharmaceutical composition for inducing an immune response in a patient with cancer without the use of a preliminary test, said composition comprising a CD8+ T lymphocytes and an isolated double stranded ribonucleic acid (dsRNA) molecule that inhibits the expression of HSP-27.

57. The pharmaceutical composition of claim 56, wherein one strand of said dsRNA is selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, and SEQ ID NO:11.

58. The pharmaceutical composition of claim 57 further comprising a first dsRNA with a strand comprising SEQ ID NO:9 and a second dsRNA with a strand comprising SEQ ID NO: 11.

59. An isolated double stranded ribonucleic acid (dsRNA) molecule that inhibits the expression of a target gene, said dsRNA comprising two strands wherein a first strand has a length from 19 to 28 consecutive nucleotides and is substantially identical to a sequence in said target gene and wherein a second strand is substantially complementary to said first strand, and a binding moiety that binds a 3′ end of said first strand to a 5′ end of said second strand, wherein one strand of said dsRNA comprises SEQ ID NO:3, SEQ ID NO:5, SEQ ID NO:7, SEQ ID NO:9, or SEQ ID NO: 11.

60. The isolated dsRNA of claim 59, further comprising a protein marker attached to said dsRNA.

61. The dsRNA of claim 60, wherein said marker protein is a fluorescent protein.

62. A vector comprising the dsRNA of any claims 59-61.

63. The vector of claim 62, wherein said vector is a retroviral vector or a lentiviral vector.

64. A cell line comprising the dsRNA of any of claims 59-61.

65. A non-human animal comprising the dsRNA of any of claims 59-61.

66. A method of treating an organism experiencing a hyperproliferative disorder, said method comprising administering a therapeutic amount of a composition comprising the dsRNA of any of claims 59-61.

67. The method of claim 66 wherein said hyperproliferative disorder is cancer.

68. The method of claim 67, wherein said cancer is brain cancer, ocular cancer, head and neck cancer, skin cancer, lung cancer, esophageal cancer, pancreatic cancer, stomach cancer, liver cancer, prostate cancer, colon cancer, rectal cancer, breast cancer, ovarian cancer, uterine cancer, cervical cancer, lymphoma, leukemia, bladder cancer or testicular cancer.

69. A pharmaceutical composition comprising the dsRNA of any of claims 59-61.

70. A pharmaceutical composition comprising multiple dsRNA of any of claims 59-61.

71. The pharmaceutical composition of claim 69 further comprising a delivery system and a tumor targeting moiety.

72. The pharmaceutical composition of claim 71, wherein said delivery system comprises a liposome, Lipid-Based Nanovectors, liposomes/lipoplexes, stable nucleic acid lipid particles and lipidoids, Biodegradable Polymeric Nanoparticles, natural polymers, including, cyclodextrin, chitosan, atelocollagen particles, synthetic polymers, including, polyethyleneimine (PEI), poly(dl-lactide-co-glycolide) (PLGA), dendrimers, Inorganic Nanoparticles, Carbon nanotubes (CNT), metals such as superparamagnetic iron oxide nanoparticles (SPION), semiconductor quantum dots (QD), manganese-doped zinc sulfide (Mn:ZnS), Gold nanoparticles (AuNP), Magnetic Nanoparticles, superparamagnetic iron oxide nanoparticles (SPIO) and magnetic iron tetroxide particles.

73. The pharmaceutical composition of claim 71, wherein said tumor targeting moiety comprises an antibody, transferrin, antibodies targeting breast cancer, Human Epidermal growth factor Receptor 2 (HER2), Epidermal Growth Factor Receptor (EGFR), Carcinoembryonic antigen (CEA), Cetuximab (C225), CD105 antibody, Gastrin releasing peptide-receptor (GRP-r), antibodies targeting Cancer Stem Cells (CSC), CD44, CD24, antibodies targeting prostate cancer, Gastrin releasing peptide-receptor (GRP-r), Anti-PSA antibody, antibodies targeting lymph node metastases, Gastrin releasing peptide-receptor (GRP-r), Anti-podoplanin antibody (PodAb), antibodies targeting pancreatic cancer cells, Neutrophil gelatinase-associated lipocalin (NGAL), mAb-F19 or combinations thereof.

74. An isolated DNA molecule that encodes an RNA that inhibits the expression of Hsp27, wherein said DNA comprises SEQ ID NO:2, SEQ ID NO:4, SEQ ID NO:6, SEQ ID NO:8, or SEQ ID NO: 10.

75. The method of claim 1, wherein one strand of said dsRNA comprises SEQ ID NO:5.

76. The method of claim 1, wherein one strand of said dsRNA comprises SEQ ID NO:7.

77. The method of claim 1, wherein one strand of said dsRNA comprises SEQ ID NO:9.

78. The method of claim 1, wherein one strand of said dsRNA comprises SEQ ID NO:11.

79. The method of claim 14, wherein said dsRNA has a length of 19 to 28 consecutive nucleotides and wherein one strand of said dsRNA comprises SEQ ID NO:5.

80. The method of claim 14, wherein said dsRNA has a length of 19 to 28 consecutive nucleotides and wherein one strand of said dsRNA comprises SEQ ID NO:7.

81. The method of claim 14, wherein said dsRNA has a length of 19 to 28 consecutive nucleotides and wherein one strand of said dsRNA comprises SEQ ID NO:9.

82. The method of claim 14, wherein said dsRNA has a length of 19 to 28 consecutive nucleotides and wherein one strand of said dsRNA comprises SEQ ID NO:11.

83. The method of claim 22, wherein said dsRNA has a length of 19 to 28 consecutive nucleotides and wherein one strand of said dsRNA comprises SEQ ID NO:5.

84. The method of claim 22, wherein said dsRNA has a length of 19 to 28 consecutive nucleotides and wherein one strand of said dsRNA comprises SEQ ID NO:7.

85. The method of claim 22, wherein said dsRNA has a length of 19 to 28 consecutive nucleotides and wherein one strand of said dsRNA comprises SEQ ID NO:9.

86. The method of claim 22, wherein said dsRNA has a length of 19 to 28 consecutive nucleotides and wherein one strand of said dsRNA comprises SEQ ID NO:11.

87. A composition comprising an isolated dsRNA molecule with a length of 19 to 28 consecutive nucleotides, which inhibits the expression of Hsp-27 using ribonucleic acid interference (RNAi) technology, and a synergistically effective amount of a platinum-containing chemotherapeutic agent, wherein one strand of said dsRNA comprises SEQ ID NO:5.

88. A composition comprising an isolated dsRNA molecule with a length of 19 to 28 consecutive nucleotides, which inhibits the expression of Hsp-27 using ribonucleic acid interference (RNAi) technology, and a synergistically effective amount of a platinum-containing chemotherapeutic agent, wherein one strand of said dsRNA comprises SEQ ID NO:7.

89. A composition comprising an isolated dsRNA molecule with a length of 19 to 28 consecutive nucleotides, which inhibits the expression of Hsp-27 using ribonucleic acid interference (RNAi) technology, and a synergistically effective amount of a platinum-containing chemotherapeutic agent, wherein one strand of said dsRNA comprises SEQ ID NO:9.

90. A composition comprising an isolated dsRNA molecule with a length of 19 to 28 consecutive nucleotides, which inhibits the expression of Hsp-27 using ribonucleic acid interference (RNAi) technology, and a synergistically effective amount of a platinum-containing chemotherapeutic agent, wherein one strand of said dsRNA comprises SEQ ID NO:11.

91. The composition of claim 87, wherein said platinum-containing chemotherapeutic agent is cisplatin, carboplatin, or oxaliplatin.

92. The composition of claim 88, wherein said platinum-containing chemotherapeutic agent is cisplatin, carboplatin, or oxaliplatin.

93. The composition of claim 89, wherein said platinum-containing chemotherapeutic agent is cisplatin, carboplatin, or oxaliplatin.

94. The composition of claim 90, wherein said platinum-containing chemotherapeutic agent is cisplatin, carboplatin, or oxaliplatin.

95. A composition comprising an isolated dsRNA molecule with a length of 19 to 28 consecutive nucleotides, which inhibits the expression of Hsp-27 using ribonucleic acid interference (RNAi) technology, and a synergistically effective amount of a topoisomerase 1 inhibitor, wherein one strand of said dsRNA comprises SEQ ID NO:5.

96. A composition comprising an isolated dsRNA molecule with a length of 19 to 28 consecutive nucleotides, which inhibits the expression of Hsp-27 using ribonucleic acid interference (RNAi) technology, and a synergistically effective amount of a topoisomerase 1 inhibitor, wherein one strand of said dsRNA comprises SEQ ID NO:7.

97. A composition comprising an isolated dsRNA molecule with a length of 19 to 28 consecutive nucleotides, which inhibits the expression of Hsp-27 using ribonucleic acid interference (RNAi) technology, and a synergistically effective amount of a topoisomerase 1 inhibitor, wherein one strand of said dsRNA comprises SEQ ID NO:9.

98. A composition comprising an isolated dsRNA molecule with a length of 19 to 28 consecutive nucleotides, which inhibits the expression of Hsp-27 using ribonucleic acid interference (RNAi) technology, and a synergistically effective amount of a topoisomerase 1 inhibitor, wherein one strand of said dsRNA comprises SEQ ID NO:11.

99. The composition of claim 95, wherein said topoisomerase 1 inhibitor is irinotecan, topotecan, camptothecin, or lamellarin D.

100. The composition of claim 96, wherein said topoisomerase 1 inhibitor is irinotecan, topotecan, camptothecin, or lamellarin D.

101. The composition of claim 97, wherein said topoisomerase 1 inhibitor is irinotecan, topotecan, camptothecin, or lamellarin D.

102. The composition of claim 98, wherein said topoisomerase 1 inhibitor is irinotecan, topotecan, camptothecin, or lamellarin D.
